(19) United States
(12) Patent Application Publication
(10) Pub. No.: US 2024/0299469 A1
(43) Pub. Date: Sep. 12, 2024

(54) PRODUCTION OF INDOLE-DERIVED METABOLITES BY SHELF-STABLE COMMUNITIES OF SAFE BACTERIA

(71) Applicant: Imvela Corp., Brooklyn, NY (US)

(72) Inventors: Dominic Colosimo, Brooklyn, NY (US); Emily Johnson, Brooklyn, NY (US); Maria Elena De Obaldia, Brooklyn, NY (US)

(73) Assignee: Imvela Corp., Brooklyn, NY (US)

(21) Appl. No.: 18/600,406

(22) Filed: Mar. 8, 2024

Related U.S. Application Data
(60) Provisional application No. 63/489,399, filed on Mar. 9, 2023.

(51) Int. Cl. A61K 35/742; A61K 47/36; A61K 47/46; A61P 37/08; C12P 17/10

(52) U.S. Cl. CPC A61K 35/742; A61K 47/36; A61K 47/46; A61P 37/08; C12P 17/10

(57) ABSTRACT

Provided herein are compositions comprising bacterial strains capable of metabolizing L-tryptophan to produce indole-containing metabolites, nutritional supplements and food products containing such bacterial strains, and methods of production thereof.

### RELATED APPLICATION

This application claims the benefit under 35 U.S.C. § 119(e) of U.S. Provisional Application No. 63/489,399, filed Mar. 9, 2023, the contents of which are incorporated by reference herein in their entirety.

### GOVERNMENT LICENSE RIGHTS

This invention was made with government support under Award #2054208 awarded by the National Science Foundation (NSF). The government has certain rights in the invention.

### REFERENCE TO AN ELECTRONIC SEQUENCE LISTING

The contents of the electronic sequence listing (1055870003US01-SEQ-CEW.xml; Size: 11,630 bytes; and Date of Creation: Mar. 8, 2024) is herein incorporated by reference in its entirety.

### BACKGROUND

Ingestion of safe bacteria with the intention of benefiting one's health is a centuries old practice that has seen a surge of interest in the early 21st century due to a better mechanistic understanding of the influence of the microbiome on organismal health. Application of these beneficial microbes, such as probiotics, is now being used in companion animals, namely canines, with the aim to modulate immune responses to combat undesirable symptoms e.g., itching, diarrhea, vomiting. The intended mechanism of this application is that probiotics with demonstrated immunomodulatory functionality can maintain viability, as measured by canonical quantitative techniques, throughout manufacturing, long-term storage, and finally ingestion by the canines to successfully engraft in the gastrointestinal mucosa and perform the desired function.

The probiotic containing products therefore require (1) relevant biological function (e.g., immune regulation), (2) viability stability amidst diverse environmental factors experienced during manufacturing, storage, and in vivo digestion, (3) voluntary/enjoyable consumption by the animal, and finally (4) key characteristics to ensure both engraftment and performance of the desired biological function.

### SUMMARY

The disclosure relates to compositions and methods for reducing inflammation in mammals, such as companion dogs. Provided herein are bacterial compositions comprising combinations of bacterial strains, such as bacteria of the genus Bacillus (e.g., Bacillus subtilis and Bacillus velezensis), for producing indole-containing metabolites (e.g., indole-3-lactic acid, indole-3-aldehyde, indole-3-acetic acid, indole-3-ethanol (IE or tryptophol) and/or indolepropionic acid) in the presence of free L-tryptophan. Without wishing to be bound by any particular theory, such indole-containing molecules are thought to act as agonists of the aryl hydrocarbon receptor (AhR), with signal transduction following stimulation having immunoregulatory effects and controlling inflammation. Indole-containing metabolites may be produced by chemical modifications (e.g., by bacterial enzymes) of free L-tryptophan, but the abundance of L-tryptophan may vary depending on the diet of a subject. In some embodiments, the compositions described herein further include one or more bacterial strains that metabolize proteins into free amino acids, including L-tryptophan, which can then be metabolized to produce indole-containing molecules that may be AhR agonists, for example indole-3-lactic acid, indole-3-aldehyde, indole-3-acetic acid, indole-3-ethanol, and/or indolepropionic acid at concentrations within the reported ranges of AhR EC₅₀s. Without wishing to be bound by a particular theory, the compositions described herein are useful for production of metabolites that stimulate AhRs, particularly canine AhR, thereby promoting homeostasis by alleviating inflammation and promoting tissue barrier integrity.

Moreover, the bacterial strains of the compositions described herein are capable of forming spores, in which they are resistant to heat, ultraviolet light, osmotic stress, low pH, enzymatic degradation, and other environmental factors. Spore formation by bacterial strains allows for the compositions described herein to maintain viability across a range of formulations, storage conditions, and administration routes, maintaining efficacy once the bacteria reach the gastrointestinal tract. In food products and nutritional supplements, in particular, spore formation allows resistance of bacterial strains to osmotic stress (and consequent maintenance of viability) at higher levels of water activity (i.e., a_{w}<0.25) necessary to avoid undesired dryness and texture. Bacteria in spore form may thus be incorporated into shelf-stable, sensorially desirable edible products, while maintaining viability through the gastrointestinal tract to produce indole-containing molecules in this niche.

Accordingly, the disclosure provides, in some aspects, a composition comprising a bacterial mixture comprising: (i) a first bacterial strain comprising a 16S rDNA sequence having at least 97% sequence identity to the nucleic acid sequence of SEQ ID NO: 1; (ii) a second bacterial strain comprising a 16S rDNA sequence having at least 97% sequence identity to the nucleic acid sequence of SEQ ID NO: 2; and (iii) a third bacterial strain comprising a 16S rDNA sequence having at least 97% sequence identity to the nucleic acid sequence of SEQ ID NO: 3, wherein the first bacterial strain and third bacterial strain are different bacterial strains.

In some embodiments, the bacterial mixture consists of: (i) a first bacterial strain comprising a 16S rDNA sequence having at least 97% sequence identity to the nucleic acid sequence of SEQ ID NO: 1; (ii) a second bacterial strain comprising a 16S rDNA sequence having at least 97% sequence identity to the nucleic acid sequence of SEQ ID NO: 2; and (iii) a third bacterial strain comprising a 16S rDNA sequence having at least 97% sequence identity to the nucleic acid sequence of SEQ ID NO: 3.

In some embodiments, (i) the first bacterial strain comprises a 16S rDNA sequence having the nucleic acid sequence of SEQ ID NO: 1; (ii) the second bacterial strain comprises a 16S rDNA sequence having the nucleic acid sequence of SEQ ID NO: 2; and (iii) the third bacterial strain comprises a 16S rDNA sequence having the nucleic acid sequence of SEQ ID NO: 3.

In some aspects, the disclosure relates to a composition comprising a bacterial mixture comprising: (i) a first bacterial strain comprising a 16S rDNA sequence having at least 97% sequence identity to the nucleic acid sequence of SEQ ID NO: 4; (ii) a second bacterial strain comprising a 16S rDNA sequence having at least 97% sequence identity to the nucleic acid sequence of SEQ ID NO: 5; and (iii) a third bacterial strain comprising a 16S rDNA sequence having at least 97% sequence identity to the nucleic acid sequence of SEQ ID NO: 6, wherein the first bacterial strain and third bacterial strain are different bacterial strains.

In some embodiments, the bacterial mixture consists of: (i) a first bacterial strain comprising a 16S rDNA sequence having at least 97% sequence identity to the nucleic acid sequence of SEQ ID NO: 4; (ii) a second bacterial strain comprising a 16S rDNA sequence having at least 97% sequence identity to the nucleic acid sequence of SEQ ID NO: 5; and (iii) a third bacterial strain comprising a 16S rDNA sequence having at least 97% sequence identity to the nucleic acid sequence of SEQ ID NO: 6.

In some embodiments, (i) the first bacterial strain comprises a 16S rDNA sequence having the nucleic acid sequence of SEQ ID NO: 4; (ii) the second bacterial strain comprises a 16S rDNA sequence having the nucleic acid sequence of SEQ ID NO: 5; and (iii) the third bacterial strain comprises a 16S rDNA sequence having the nucleic acid sequence of SEQ ID NO: 6.

In some aspects, the disclosure relates to a composition comprising a bacterial mixture comprising a first bacterial strain belonging to the species Bacillus subtilis, a second bacterial strain belonging to the species Bacillus velezensis, and a third bacterial strain belonging to the species Bacillus subtilis, wherein the first bacterial strain and third bacterial strain are different bacterial strains.

In some embodiments, the bacterial mixture consists of a first bacterial strain belonging to the species Bacillus subtilis, a second bacterial strain belonging to the species Bacillus velezensis, and a third bacterial strain belonging to the species Bacillus subtilis.

In some embodiments, the composition further comprises one or more additional bacterial strains. In some embodiments, one or more of the additional bacterial strains belong to the genus Bacillus. In some embodiments, one or more of the additional bacterial strains belong to the species Bacillus subtilis, Bacillus velezensis, Bacillus amyloliquefaciens, Bacillus lentus, Bacillus licheniformis, or Bacillus pumilus.

In some embodiments, the one or more of the additional bacterial strains comprise a 16S rDNA sequence with at least 97% sequence identity to the nucleic acid sequence of any one of SEQ ID NOs: 1-3. In some embodiments, one or more of the additional bacterial strains comprise a 16S rDNA sequence with at least 97% sequence identity to the nucleic acid sequence of any one of SEQ ID NOs; 4-6.

In some embodiments, the composition produces one or more indole-containing molecules in the presence of L-tryptophan. In some embodiments, the composition produces at least 4 indole-containing molecules in the presence of L-tryptophan. In some embodiments, the indole-containing molecules are indoleacetic acid, indoleacrylic acid, indolepropionic acid, indolepyruvic acid, indoleacetamide, indole-3-acetic acid, indole-3-ethanol, indolealdehyde, indole, and/or tryptamine. In some embodiments, the indole-containing molecules comprise indole-3-lactic acid, indole-3-aldehyde, indolepropionic acid, and/or indole-3-acetic acid.

In some embodiments, the composition produces indole-3-acetic acid in a concentration of 8.5 M or more. In some embodiments, the composition produces indoleacetic acid in a concentration of 10 μM or more. In some embodiments, the composition produces indolepropionic acid in a concentration of 1.0 μM or more. In some embodiments, the composition produces indole-3-aldehyde in a concentration of 1.0 μM or more.

In some embodiments, the composition produces L-tryptophan in the presence of protein. In some embodiments, the composition reduces inflammation, or a symptom thereof, in a subject, e.g., following administration of the composition to the subject. In some embodiments, the composition treats or prevents a symptom associated with inflammation in a subject. In some embodiments, the composition treats or prevents diarrhea in a subject. In some embodiments, the composition treats or prevents vomiting in a subject. In some embodiments, the composition treats or prevents itching, paw licking, and/or head shaking in a subject. In some embodiments, the composition treats or prevents allergy in a subject.

In some embodiments, the subject is a carnivore. In some embodiments, the subject is a mammal. In some embodiments, the subject is a domesticated animal. In some embodiments, the subject is a rodent. In some embodiments, the subject is a mouse, rat, guinea pig, chinchilla, or hamster. In some embodiments, the subject is a dog, cat, rabbit, guinea pig, hamster, or ferret. In some embodiments, the subject is a dog. In some embodiments, the subject is a bovine, swine, llama, alpaca, sheep, or goat. In some embodiments, the subject is a bird.

In some embodiments, the bacterial strains are lyophilized. In some embodiments, the bacterial strains are spray-dried. In some embodiments, the bacterial strains are in a dry, powdered form.

In some embodiments, one or more of the bacterial strains is in spore form. In some embodiments, each of the bacterial strains is in spore form. In some embodiments, one or more of the bacterial strains is in vegetative form. In some embodiments, each of the bacterial strains is in vegetative form.

In some embodiments, the composition has a water activity level (a_{w}) of 0.8 or less. In some embodiments, at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the composition are viable after storage for a period of time. In some embodiments, the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.

In some embodiments, the storage is conducted at 2° C. to 8° C., 8° C. to 20° C., or 20° C. to 40° C. In some embodiments, the storage is conducted at a temperature of 25° C. or higher.

In some embodiments, the composition has a water activity level (a_{w}) of 0.25 or more, 0.3 or more, 0.4 or more, 0.5 or more, 0.6 or more, or 0.7 or more during the storage.

In some embodiments, the compositions further comprises one or more pH-sensitive enteric polymers.

In some embodiments, each of the bacterial strains is present in an amount from 10⁵ to 10¹³ colony forming units (CFUs) per gram of the composition. In some embodiments, each of the bacterial strains is present in an amount from 10⁵ to 10⁸ CFUs per gram of the composition.

In some aspects, the disclosure relates to a nutritional supplement comprising the composition as described herein, and a carrier. In some embodiments, each of the bacterial strains is present in an amount from 10⁵ to 10¹³ colony forming units (CFUs) per gram of the nutritional supplement. In some embodiments, each of the bacterial strains is present in an amount from 10⁸ to 10¹⁰ CFUs per gram of the nutritional supplement.

In some embodiments, the nutritional supplement has a water activity (a_{w}) of 0.8 or less.

In some embodiments, at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the nutritional supplement are viable after storage for a period of time. In some embodiments, the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.

In some embodiments, the storage is conducted at 2° C. to 8° C., 8° C. to 20° C., or 20° C. to 40° C. In some embodiments, the storage is conducted at a temperature of 25° C. or more.

In some embodiments, the nutritional supplement has a water activity level (a_{w}) of 0.25 or more, 0.3 or more, 0.4 or more, 0.5 or more, 0.6 or more, or 0.7 or more during the storage.

In some embodiments, the nutritional supplement is a canine nutritional supplement. In some embodiments, the nutritional supplement comprises a meat or animal-derived material. In some embodiments, the meat or animal-derived material is beef, pork, chicken, eggs, turkey, lamb, fish, blood marrow, and/or bone marrow. In some embodiments, the nutritional supplement comprises a grain. In some embodiments, the grain is wheat, corn, rice, oats, and/or barley.

In some embodiments, the nutritional supplement comprises a fiber. In some embodiments, the fiber is sugar beet pulp, chicory pulp, chicory, coconut endosperm fiber, wheat fiber, fructooligosaccharides, galactooligosaccharides, and/or inulin.

In some embodiments, the nutritional supplement comprises a gelatinized starch matrix.

In some embodiments, the nutritional supplement reduces inflammation in a subject. In some embodiments, the nutritional supplement treats or prevents a symptom associated with inflammation in a subject. In some embodiments, the nutritional supplement treats or prevents diarrhea in a subject. In some embodiments, the nutritional supplement treats or prevents vomiting in a subject. In some embodiments, the nutritional supplement treats or prevents itching, paw licking, and/or head shaking in a subject.

In some aspects, the disclosure relates to method for producing a nutritional supplement, the method comprising: (i) providing a carrier; and (ii) adding a composition comprising a bacterial mixture to the carrier to produce the nutritional supplement, wherein the bacterial mixture comprises: (a) a first bacterial strain comprising a 16S rDNA sequence having at least 97% sequence identity to the nucleic acid sequence of SEQ ID NO: 1; (b) a second bacterial strain comprising a 16S rDNA sequence having at least 97% sequence identity to the nucleic acid sequence of SEQ ID NO: 2; and (c) a third bacterial strain comprising a 16S rDNA sequence having at least 97% sequence identity to the nucleic acid sequence of SEQ ID NO: 3; wherein the first bacterial strain and third bacterial strain are different bacterial strains.

In some embodiments, the bacterial mixture consists of: (i) the first bacterial strain comprising a 16S rDNA sequence having at least 97% sequence identity to the nucleic acid sequence of SEQ ID NO: 1; (ii) the second bacterial strain comprising a 16S rDNA sequence having at least 97% sequence identity to the nucleic acid sequence of SEQ ID NO: 2; and (iii) the third bacterial strain comprising a 16S rDNA sequence having at least 97% sequence identity to the nucleic acid sequence of SEQ ID NO: 3.

In some embodiments, (i) the first bacterial strain comprises a 16S rDNA sequence having the nucleic acid sequence of SEQ ID NO: 1; (ii) the second bacterial strain comprises a 16S rDNA sequence having the nucleic acid sequence of SEQ ID NO: 2; and (iii) the third bacterial strain comprises a 16S rDNA sequence having the nucleic acid sequence of SEQ ID NO: 3.

In some aspects, the disclosure relates to a method for producing a nutritional supplement, the method comprising: (i) providing a carrier; and (ii) adding a composition comprising a bacterial mixture to the carrier to produce the nutritional supplement, wherein the bacterial mixture comprises: (a) a first bacterial strain comprising a 16S rDNA sequence having at least 97% sequence identity to the nucleic acid sequence of SEQ ID NO: 4; (b) a second bacterial strain comprising a 16S rDNA sequence having at least 97% sequence identity to the nucleic acid sequence of SEQ ID NO: 5; and (c) a third bacterial strain comprising a 16S rDNA sequence having at least 97% sequence identity to the nucleic acid sequence of SEQ ID NO: 6; wherein the first bacterial strain and third bacterial strain are different bacterial strains.

In some embodiments, wherein the bacterial mixture consists of: (i) the first bacterial strain comprising a 16S rDNA sequence having at least 97% sequence identity to the nucleic acid sequence of SEQ ID NO: 4; (ii) the second bacterial strain comprising a 16S rDNA sequence having at least 97% sequence identity to the nucleic acid sequence of SEQ ID NO: 5; and (iii) the third bacterial strain comprising a 16S rDNA sequence having at least 97% sequence identity to the nucleic acid sequence of SEQ ID NO: 6.

In some embodiments, (i) the first bacterial strain comprises a 16S rDNA sequence having the nucleic acid sequence of SEQ ID NO: 4; (ii) the second bacterial strain comprises a 16S rDNA sequence having the nucleic acid sequence of SEQ ID NO: 5; and (iii) the third bacterial strain comprises a 16S rDNA sequence having the nucleic acid sequence of SEQ ID NO: 6.

In some aspects, the disclosure relates to a method for producing a nutritional supplement, the method comprising: (i) providing a carrier; and (ii) adding a composition comprising a bacterial mixture to the carrier to produce the nutritional supplement, wherein the bacterial mixture comprises a first bacterial strain belonging to the species Bacillus subtilis, a second bacterial strain belonging to the species Bacillus velezensis, and a third bacterial strain belonging to the species Bacillus subtilis, wherein the first bacterial strain and the third bacterial strain are different bacterial strains.

In some embodiments, the bacterial mixture consists of a first bacterial strain belong to the species Bacillus subtilis, a second bacterial strain belongs to the species Bacillus velezensis, and a third bacterial strain belong to the species Bacillus subtilis. In some embodiments, the bacterial mixture further comprises one or more additional bacterial strains. In some embodiments, one or more of the additional bacterial strains belong to the genus Bacillus. In some embodiments, one or more of the additional bacterial strains belong to the species Bacillus subtilis, Bacillus velezensis, Bacillus amyloliquefaciens, Bacillus lentus, Bacillus licheniformis, or Bacillus pumilus.

In some embodiments, one or more of the additional bacterial strains comprise a 16S rDNA sequence with at least 97% sequence identity to the nucleic acid sequence of any one of SEQ ID NOs: 1-3. In some embodiments, one or more of the additional bacterial strains comprise a 16S rDNA sequence with at least 97% sequence identity to the nucleic acid sequence of any one of SEQ ID NOs; 4-6.

In some embodiments, the composition produces one or more indole-containing molecules in the presence of L-tryptophan. In some embodiments, the composition produces at least 4 indole-containing molecules in the presence of L-tryptophan. In some embodiments, the indole-containing molecules comprise indoleacetic acid, indoleacrylic acid, indolepropionic acid, indolepyruvic acid, indoleacetamide, indole-3-acetic acid, indole-3-ethanol, indolealdehyde, indole, and/or tryptamine.

In some embodiments, the composition produces indole-3-acetic acid in a concentration of 8.5 UM or more. In some embodiments, the composition produces indoleacetic acid in a concentration of 10 μM or more. In some embodiments, the composition produces indolepropionic acid in a concentration of 1.0 μM or more. In some embodiments, the composition produces indole-3-aldehyde in a concentration of 1.0 μM or more.

In some embodiments, the composition produces L-tryptophan in the presence of protein.

In some embodiments, the composition reduces inflammation in a subject. In some embodiments, the composition treats or prevents a symptom associated with inflammation in a subject. In some embodiments, the composition treats or prevents diarrhea in a subject. In some embodiments, the composition treats or prevents vomiting in a subject. In some embodiments, the composition treats or prevents itching, paw licking, and/or head shaking in a subject.

In some embodiments, at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the composition are viable after storage for a period of time.

In some embodiments, the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In some embodiments, the storage is conducted at 2° C. to 8° C., 8° C. to 20° C., or 20° C. to 40° C. In some embodiments, the storage is conducted at a temperature of 25° C. or more.

In some embodiments, the composition has a water activity level (a_{w}) of 0.25 or more, 0.3 or more, 0.4 or more, 0.5 or more, 0.6 or more, or 0.7 or more during the storage.

In some embodiments, the bacterial strains are lyophilized. In some embodiments, the bacterial strains are spray-dried.

In some embodiments, one or more of the bacterial strains is in spore form. In some embodiments, each of the bacterial strains is in spore form. In some embodiments, one or more of the bacterial strains is in vegetative form. In some embodiments, each of the bacterial strains is in vegetative form.

In some embodiments, the composition has a water activity level (a_{w}) of 0.8 or less. In some embodiments, the composition further comprises one or more pH-sensitive enteric polymers.

In some embodiments, each of the bacterial strains is present in an amount from 10⁵ to 10¹³ colony forming units (CFUs) per gram of the composition. In some embodiments, each of the bacterial strains is present in an amount from 10⁵ to 10⁸ CFUs per gram of the nutritional supplement. In some embodiments, each of the bacterial strains is present in an amount from 10⁵ to 10¹³ colony forming units (CFUs) per gram of the nutritional supplement.

In some embodiments, each of the bacterial strains is present in an amount from 10⁸ to 10¹⁰ CFUs per gram of the nutritional supplement.

In some embodiments, the nutritional supplement has a water activity (a_{w}) of 0.8 or less.

In some embodiments, at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the nutritional supplement are viable after storage for a period of time.

In some embodiments, the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.

In some embodiments, the storage is conducted at 2° C. to 8° C., 8° C. to 20° C., or 20° C. to 40° C. In some embodiments, the storage is conducted at a temperature of 25° C. or more.

In some embodiments, the nutritional supplement has a water activity level (a_{w}) of 0.25 or more, 0.3 or more, 0.4 or more, 0.5 or more, 0.6 or more, or 0.7 or more during the storage.

In some embodiments, the nutritional supplement is a canine nutritional supplement.

In some embodiments, the nutritional supplement comprises a meat or animal-derived material. In some embodiments, the meat or animal-derived material is beef, pork, chicken, eggs, turkey, lamb, fish, blood marrow, and/or bone marrow. In some embodiments, the nutritional supplement comprises a grain. In some embodiments, the grain is wheat, corn, rice, oats, and/or barley. In some embodiments, the nutritional supplement comprises a fiber. In some embodiments, the fiber is sugar beet pulp, chicory pulp, chicory, coconut endosperm fiber, wheat fiber, fructooligosaccharides, galactooligosaccharides, and/or inulin.

In some embodiments, the nutritional supplement comprises a gelatinized starch matrix.

In some embodiments, the nutritional supplement reduces inflammation in a subject. In some embodiments, the nutritional supplement treats or prevents a symptom associated with inflammation in a subject. In some embodiments, the nutritional supplement treats or prevents diarrhea in a subject. In some embodiments, the nutritional supplement treats or prevents vomiting in a subject. In some embodiments, the nutritional supplement treats or prevents itching, paw licking, and/or head shaking in a subject. In some embodiments, the nutritional supplement treats or prevents one or more symptoms associated with allergy in a subject.

In some aspects, the disclosure relates to a nutritional supplement produced by a method as described herein.

In some aspects, the disclosure relates to a food product comprising a composition as described herein, and a nutrient.

In some embodiments, each of the bacterial strains is present in an amount from 10⁵ to 10¹³ colony forming units (CFUs) per gram of the food product. In some embodiments, each of the bacterial strains is present in an amount from 10⁸ to 1010 CFUs per gram of the food product.

In some embodiments, the food product has a water activity (a_{w}) of 0.8 or less.

In some embodiments, at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the food product are viable after storage for a period of time. In some embodiments, the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In some embodiments, the storage is conducted at 2° C. to 8° C., 8° C. to 20° C., or 20° C. to 40° C. In some embodiments, the storage is conducted at a temperature of 25° C. or more.

In some embodiments, the food product has a water activity level (a_{w}) of 0.25 or more, 0.3 or more, 0.4 or more, 0.5 or more, 0.6 or more, or 0.7 or more during the storage.

In some embodiments, the food product is a canine food product. In some embodiments, the food product comprises a meat or animal-derived material. In some embodiments, the meat or animal-derived material is beef, pork, chicken, eggs, turkey, lamb, fish, blood marrow, and/or bone marrow. In some embodiments, the food product comprises a grain. In some embodiments, the grain is wheat, corn, rice, oats, and/or barley. In some embodiments, the food product comprises a fiber. In some embodiments, the fiber is sugar beet pulp, chicory pulp, chicory, coconut endosperm fiber, wheat fiber, fructooligosaccharides, galactooligosaccharides, and/or inulin. In some embodiments, the food product comprises a gelatinized starch matrix.

In some embodiments, the food product reduces inflammation in a subject. In some embodiments, the food product treats or prevents a symptom associated with inflammation in a subject. In some embodiments, the food product treats or prevents diarrhea in a subject. In some embodiments, the food product treats or prevents vomiting in a subject. In some embodiments, the food product treats or prevents itching, paw licking, and/or head shaking in a subject.

In some aspects, the disclosure relates to a method for producing a food product, the method comprising: (i) providing a carrier; and (ii) adding a composition comprising a bacterial mixture to the nutrient to produce the food product, wherein the bacterial mixture comprises: (a) a first bacterial strain comprising a 16S rDNA sequence having at least 97% sequence identity to the nucleic acid sequence of SEQ ID NO: 1; (b) a second bacterial strain comprising a 16S rDNA sequence having at least 97% sequence identity to the nucleic acid sequence of SEQ ID NO: 2; and (c) a third bacterial strain comprising a 16S rDNA sequence having at least 97% sequence identity to the nucleic acid sequence of SEQ ID NO: 3; wherein the first bacterial strain and third bacterial strain are different bacterial strains.

In some embodiments, the bacterial mixture consists of: (i) the first bacterial strain comprising a 16S rDNA sequence having at least 97% sequence identity to the nucleic acid sequence of SEQ ID NO: 1; (ii) the second bacterial strain comprising a 16S rDNA sequence having at least 97% sequence identity to the nucleic acid sequence of SEQ ID NO: 2; and (iii) the third bacterial strain comprising a 16S rDNA sequence having at least 97% sequence identity to the nucleic acid sequence of SEQ ID NO: 3.

In some embodiments, (i) the first bacterial strain comprises a 16S rDNA sequence having the nucleic acid sequence of SEQ ID NO: 1; (ii) the second bacterial strain comprises a 16S rDNA sequence having the nucleic acid sequence of SEQ ID NO: 2; and (iii) the third bacterial strain comprises a 16S rDNA sequence having the nucleic acid sequence of SEQ ID NO: 3.

In some aspects, the disclosure relates to a method for producing a food product, the method comprising: (i) providing a carrier; and (ii) adding a composition comprising a bacterial mixture to the carrier to produce the food product, wherein the bacterial mixture comprises: (a) a first bacterial strain comprising a 16S rDNA sequence having at least 97% sequence identity to the nucleic acid sequence of SEQ ID NO: 4; (b) a second bacterial strain comprising a 16S rDNA sequence having at least 97% sequence identity to the nucleic acid sequence of SEQ ID NO: 5; and (c) a third bacterial strain comprising a 16S rDNA sequence having at least 97% sequence identity to the nucleic acid sequence of SEQ ID NO: 6; wherein the first bacterial strain and third bacterial strain are different bacterial strains.

In some embodiments, the bacterial mixture consists of: (i) the first bacterial strain comprising a 16S rDNA sequence having at least 97% sequence identity to the nucleic acid sequence of SEQ ID NO: 4; (ii) the second bacterial strain comprising a 16S rDNA sequence having at least 97% sequence identity to the nucleic acid sequence of SEQ ID NO: 5; and (iii) the third bacterial strain comprising a 16S rDNA sequence having at least 97% sequence identity to the nucleic acid sequence of SEQ ID NO: 6. In some embodiments, (i) the first bacterial strain comprises a 16S rDNA sequence having the nucleic acid sequence of SEQ ID NO: 4; (ii) the second bacterial strain comprises a 16S rDNA sequence having the nucleic acid sequence of SEQ ID NO: 5; and (iii) the third bacterial strain comprises a 16S rDNA sequence having the nucleic acid sequence of SEQ ID NO: 6.

In some aspects, the disclosure relates to a method for producing a food product, the method comprising: (i) providing a nutrient; and (ii) adding a composition comprising a bacterial mixture to the nutrient to produce the food product, wherein the bacterial mixture comprises a first bacterial strain belonging to the species Bacillus subtilis, a second bacterial strain belonging to the species Bacillus velezensis, and a third bacterial strain belonging to the species Bacillus subtilis, wherein the first bacterial strain and the third bacterial strain are different bacterial strains. In some embodiments, the bacterial mixture consists of a first bacterial strain belong to the species Bacillus subtilis, a second bacterial strain belongs to the species Bacillus velezensis, and a third bacterial strain belong to the species Bacillus subtilis.

In some embodiments, the bacterial mixture further comprises one or more additional bacterial strains. In some embodiments, one or more of the additional bacterial strains belong to the genus Bacillus.

In some embodiments, one or more of the additional bacterial strains belong to the species Bacillus subtilis, Bacillus velezensis, Bacillus amyloliquefaciens, Bacillus lentus, Bacillus licheniformis, or Bacillus pumilus. In some embodiments, one or more of the additional bacterial strains comprise a 16S rDNA sequence with at least 97% sequence identity to the nucleic acid sequence of any one of SEQ ID NOs: 1-3. In some embodiments, one or more of the additional bacterial strains comprise a 16S rDNA sequence with at least 97% sequence identity to the nucleic acid sequence of any one of SEQ ID NOs; 4-6.

In some embodiments, the composition produces one or more indole-containing molecules in the presence of L-tryptophan. In some embodiments, the composition produces at least 4 indole-containing molecules in the presence of L-tryptophan. In some embodiments, the indole-containing molecules comprise indoleacetic acid, indoleacrylic acid, indolepropionic acid, indolepyruvic acid, indoleacetamide, indole-3-acetic acid, indole-3-ethanol, indolealdehyde, indole, and/or tryptamine.

In some embodiments, the composition produces indole-3-acetic acid in a concentration of 8.5 μM or more. In some embodiments, the composition produces indoleacetic acid in a concentration of 10 μM or more. In some embodiments, the composition produces indolepropionic acid in a concentration of 1.0 μM or more. In some embodiments, the composition produces indole-3-aldehyde in a concentration of 1.0 μM or more.

In some embodiments, the composition produces L-tryptophan in the presence of protein.

In some embodiments, the composition reduces inflammation in a subject. In some embodiments, the composition treats or prevents a symptom associated with inflammation in a subject. In some embodiments, the composition treats or prevents diarrhea in a subject. In some embodiments, the composition treats or prevents vomiting in a subject. In some embodiments, the composition treats or prevents itching, paw licking, and/or head shaking in a subject.

In some embodiments, at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the composition are viable after storage for a period of time.

In some embodiments, the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In some embodiments, the storage is conducted at 2° C. to 8° C., 8° C. to 20° C., or 20° C. to 40° C. In some embodiments, the storage is conducted at a temperature of 25° C. or more.

In some embodiments, the composition has a water activity level (a_{w}) of 0.25 or more, 0.3 or more, 0.4 or more, 0.5 or more, 0.6 or more, or 0.7 or more during the storage.

In some embodiments, the bacterial strains are lyophilized. In some embodiments, the bacterial strains are spray-dried. In some embodiments, one or more of the bacterial strains is in spore form. In some embodiments, each of the bacterial strains is in spore form. In some embodiments, one or more of the bacterial strains is in vegetative form. In some embodiments, each of the bacterial strains is in vegetative form.

In some embodiments, the food product has a water activity level (a_{w}) of 0.8 or less.

In some embodiments, the food product further comprises one or more pH-sensitive enteric polymers.

In some embodiments, each of the bacterial strains is present in an amount from 10⁵ to 10¹³ colony forming units (CFUs) per gram of the food product. In some embodiments, each of the bacterial strains is present in an amount from 10⁵ to 10⁸ CFUs per gram of the food product. In some embodiments, each of the bacterial strains is present in an amount from 10⁵ to 10¹³ colony forming units (CFUs) per gram of the food product. In some embodiments, each of the bacterial strains is present in an amount from 10⁸ to 1010 CFUs per gram of the food product.

In some embodiments, the food product has a water activity (a_{w}) of 0.8 or less.

In some embodiments, at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the food product are viable after storage for a period of time.

In some embodiments, the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In some embodiments, the storage is conducted at 2° C. to 8° C., 8° C. to 20° C., or 20° C. to 40° C. In some embodiments, the storage is conducted at a temperature of 25° C. or more.

In some embodiments, the food product has a water activity level (a_{w}) of 0.25 or more, 0.3 or more, 0.4 or more, 0.5 or more, 0.6 or more, or 0.7 or more during the storage.

In some embodiments, the food product is a canine food product.

In some embodiments, the food product comprises a meat or animal-derived material. In some embodiments, the meat or animal-derived material is beef, pork, chicken, eggs, turkey, lamb, fish, blood marrow, and/or bone marrow. In some embodiments, the food product comprises a grain. In some embodiments, the grain is wheat, corn, rice, oats, and/or barley. In some embodiments, the food product comprises a fiber. In some embodiments, the fiber is sugar beet pulp, chicory pulp, chicory, coconut endosperm fiber, wheat fiber, fructooligosaccharides, galactooligosaccharides, and/or inulin. In some embodiments, the food product comprises a gelatinized starch matrix.

In some embodiments, the food product reduces inflammation in a subject. In some embodiments, the food product treats or prevents a symptom associated with inflammation in a subject. In some embodiments, the food product treats or prevents diarrhea in a subject. In some embodiments, the food product treats or prevents vomiting in a subject. In some embodiments, the food product treats or prevents itching, paw licking, and/or head shaking in a subject. In some embodiments, the food product treats or prevents one or more symptoms associated with allergy in a subject.

In some aspects, the disclosure relates to a food product produced by a method as described herein.

In some aspects, the disclosure relates to a method comprising administering a composition, nutritional supplement, or food product as described herein, to a subject in need thereof. In some embodiments, the subject is a carnivore. In some embodiments, the subject is a mammal. In some embodiments, the subject is a domesticated animal. In some embodiments, the subject is a rodent. In some embodiments, the subject is a mouse, rat, guinea pig, chinchilla, or hamster. In some embodiments, the subject is a dog, cat, rabbit, guinea pig, hamster, or ferret. In some embodiments, the subject is a dog. In some embodiments, the subject is a bovine, swine, llama, alpaca, sheep, or goat. In some embodiments, the subject is a bird.

In some embodiments, the subject has inflammation, diarrhea, vomiting, and/or itching, paw licking, and/or head shaking. In some embodiments, the subject has one or more symptoms associated with allergy.

In some aspects, the disclosure provides a nutritional supplement comprising a bacterial mixture and a carrier, the bacterial mixture comprising: (i) a first bacterial strain comprising a 16S rDNA sequence with at least 97% sequence identity to the nucleotide sequence of SEQ ID NO: 1; (ii) a second bacterial strain comprising a 16S rDNA sequence with at least 97% sequence identity to the nucleotide sequence of SEQ ID NO: 2; and (iii) a third bacterial strain comprising a 16S rDNA sequence with at least 97% sequence identity to the nucleotide sequence of SEQ ID NO: 3; wherein the first and third bacterial strains are different strains.

In some embodiments, the nutritional supplement comprises a gelatinized starch matrix. In some embodiments, the nutritional supplement has a water activity (a_{w}) of 0.25 or more. In some embodiments, the carrier is maltodextrin. In some embodiments, the carrier is corn maltodextrin. In some embodiments, the nutritional supplement is in the form of a chew.

In some embodiments, the bacterial strains are lyophilized or spray-dried. In some embodiments, the bacterial strains are in spore form.

In some embodiments: (i) the first bacterial strain comprises a 16S rDNA sequence with at least 99% sequence identity to the nucleotide sequence of SEQ ID NO: 1; (ii) the second bacterial strain comprises a 16S rDNA sequence with at least 99% sequence identity to the nucleotide sequence of SEQ ID NO: 2; and (iii) the third bacterial strain comprises a 16S rDNA sequence with at least 99% sequence identity to the nucleotide sequence of SEQ ID NO: 3.

In some embodiments, the nutritional supplement comprises 3×10⁶ to 3×10⁸ colony forming units (CFUs) of each of the bacterial strains per gram of the nutritional supplement. In some embodiments, the nutritional supplement comprises 1×107 to 1×109 total colony forming units (CFUs) of the bacterial strains per gram of the nutritional supplement.

In some embodiments, at least 50% of colony forming units of the bacterial strains are viable after storage for 4 weeks. In some embodiments, the storage is conducted at a temperature of at least 25° C.

In some embodiments, the nutritional supplement produces indole-3-lactic acid (ILA), indole-3-aldehyde (IAld), and indole-3-acetic acid (IAA) in a subject. In some embodiments, wherein the ILA, IAld, and IAA are produced in an intestine of the subject. In some embodiments, the nutritional supplement: (i) reduces inflammation; (ii) treats or prevents diarrhea; (iii) treats or prevents vomiting; (iv) treats or prevents itching, paw licking, and/or head shaking; and/or (v) treats or prevents allergy, in a subject. In some embodiments, the subject is a dog.

In some embodiments, the nutritional supplement comprises a fiber selected from sugar beet pulp, chicory pulp, chicory, coconut endosperm fiber, wheat fiber, fructooligosaccharides, galactooligosaccharides, and inulin. In some embodiments, the nutritional supplement comprises a grain selected from wheat, corn, rice, oats, and barley. In some embodiments, the nutritional supplement comprises a meat or animal-derived material selected from beef, pork, chicken, eggs, turkey, lamb, fish, blood marrow, and bone marrow.

Aspects of the present disclosure provide a nutritional supplement comprising a bacterial mixture and a carrier, the bacterial mixture comprising: (i) a first bacterial strain comprising a 16S rDNA sequence with at least 97% sequence identity to the nucleotide sequence of SEQ ID NO: 1; (ii) a second bacterial strain comprising a 16S rDNA sequence with at least 97% sequence identity to the nucleotide sequence of SEQ ID NO: 2; and (iii) third bacterial strain comprising a 16S rDNA sequence with at least 97% sequence identity to the nucleotide sequence of SEQ ID NO: 3; wherein the first and third bacterial strains are different bacterial strains; and wherein the carrier comprises a gelatinized starch matrix and the nutritional supplement has a water activity (a_{w}) of 0.25 or more.

In some embodiments, the water activity (a_{w}) is between 0.4 to 0.7. In some embodiments, the carrier comprises maltodextrin. In some embodiments, the carrier comprises a starch and/or a fiber, optionally a soluble or insoluble fiber. In some embodiments, the fiber is selected from sugar beet pulp, chicory pulp, chicory, coconut endosperm fiber, wheat fiber, fructooligosaccharides, galactooligosaccharides, and inulin.

In some embodiments, the nutritional supplement is in the form of a chew. In some embodiments, the bacterial strains are lyophilized or spray-dried. In some embodiments, the bacterial strains are in spore form.

In some embodiments, the bacterial mixture consists of: (i) the first bacterial strain comprising a 16S rDNA sequence with at least 97% sequence identity to the nucleotide sequence of SEQ ID NO: 1; (ii) the second bacterial strain comprising a 16S rDNA sequence with at least 97% sequence identity to the nucleotide sequence of SEQ ID NO: 2; and (iii) the third bacterial strain comprising a 16S rDNA sequence with at least 97% sequence identity to the nucleotide sequence of SEQ ID NO: 3.

In some embodiments, the bacterial mixture comprises: (i) the first bacterial strain comprising a 16S rDNA sequence with at least 99% sequence identity to the nucleotide sequence of SEQ ID NO: 1; (ii) the second bacterial strain comprising a 16S rDNA sequence with at least 99% sequence identity to the nucleotide sequence of SEQ ID NO: 2; and (iii) the third bacterial strain comprising a 16S rDNA sequence with at least 99% sequence identity to the nucleotide sequence of SEQ ID NO: 3.

In some embodiments, the nutritional supplement comprises 1×107 to 1×109 colony forming units (CFUs) of each of the bacterial strains per gram of the nutritional supplement. In some embodiments, the nutritional supplement comprises 1×10⁸ to 1×10¹⁰ total colony forming units (CFUs) of the bacterial strains per gram of the nutritional supplement.

In some embodiments, at least 50% of colony forming units of the bacterial strains are viable after storage for 4 weeks. In some embodiments, the storage is conducted at a temperature of at least 25° C.

In some embodiments, the nutritional supplement produces indole-3-lactic acid (ILA), indole-3-aldehyde (IAld), and indole-3-acetic acid (IAA) in a subject. In some embodiments, the ILA, IAld, and IAA are produced in an intestine of the subject.

In some embodiments, the nutritional supplement treats or prevents itching and/or allergy in a subject. In some embodiments, the subject is a dog.

In some embodiments, the nutritional supplement comprises a grain selected from the group consisting of wheat, corn, rice, oats, and barley. In some embodiments, the nutritional supplement comprises a meat, optionally an animal-derived material selected from the group consisting of beef, chicken, pork, eggs, turkey, lamb, fish, blood marrow, and bone marrow.

### BRIEF DESCRIPTION OF THE DRAWINGS

The accompanying drawings, which constitute a part of this specification, illustrate several embodiments of the disclosure and together with the description, serve to explain the principles of the disclosure.

FIGS. 1A-1H demonstrate identification of microorganisms that catabolize tryptophan into specific indole-containing metabolites. FIG. 1A shows exemplary indole-containing compounds that can be produced by various microbes from the substrate L-tryptophan (center box). FIG. 1B shows growth of microbial strains under anaerobic conditions resembling those of the canine gastrointestinal tract, allowing identification of microbes that remain viable until reaching the terminal delivery site. FIG. 1C summarizes results from a high-throughput quantification of exemplary indole-containing molecules from thousands of individual bacterial strains (represented by circles). Circles not indicated for each compound indicate that the molecules were below the limit of quantification for that particular bacterial strain. Indole-3-lactic acid was observed as the most abundant indole-containing molecule detected, and indole propionic acid was found to be the least abundant molecule, among the bacterial strains tested. Bacterial strains producing the highest levels for each indole-containing molecule were identified and selected for combination experiments. FIG. 1D shows a schematic of the functionality of an exemplary bacterial combination in producing a combination of indole-containing molecules, such as indole-3-acetic acid, indolepropionic acid, indole-3-aldehyde, and indolelactic acid, with expected final concentrations of indole-containing molecules indicated for each chemotype. FIG. 1E shows the different mixtures of compounds between the 3 strains showing strain-specificity that allows for orthogonal biological effects. FIG. 1F demonstrates how several qualifying Bacillus cultures are capable of hydrolyzing a complex protein mixture into free L-tryptophan that could specifically feed the AHR agonist biosynthesis of surrounding cultures. The dashed line represents the starting free L-tryptophan in the media. The strain indicated by the arrow showed the most robust liberation of L-tryptophan from a complex protein source and was determined to be an integral member of the community as a potential substrate provider. FIG. 1G shows, for each strain, the relative amounts of indole-3-lactic acid (ILA), indole-3-aldehyde (IAld), and indole-3-acetic acid (IAA) produced. FIG. 1H shows, for each strain, the absolute amounts of ILA, IAld, and IAA produced.

FIG. 2 shows the results of cross-feeding experiments of bacterial communities, demonstrating that bacterial combinations tested provide the complete chemical repertoire for this metabolism to take place under a broad range of conditions that may be present across individual canines. Bar graphs show synergistic production of indoleacetic acid (IAA) when B. subtilis and B. velezensis are grown adjacently separated by a permeable membrane that permits diffusion of small molecules and macromolecules.

FIGS. 3A-3D shows results of stability testing, demonstrating the ability of bacterial combinations tested to survive harsh environmental conditions that are canonically prohibitive to maintenance of bacterial viability. FIGS. 3A and 3C show that each of the three strains can survive conditions mimicking gastric stress (pH 2 with endopeptidases at biological concentrations) as well as intestinal stress (neutral pH with endopeptidases and 18 g/L bile salts) with a <0.5 log₁₀ loss in viability. FIGS. 3B and 3D show that viability of Bacillus strains (black) can be maintained for >4 weeks (<0.5 log₁₀ loss in viability) in a pilot pet chew matrix with a water activity (a_{w}) of 0.4, which is growth prohibitive to conventional commercial probiotics like Lactobacillus (grey).

FIGS. 4A-4D show production of indole-containing compounds by multiple different strains of Bacillus velezensis and Bacillus subtilis. FIG. 4A shows the extent of ILA production. FIG. 4B shows the extent of IAld production. FIG. 4C shows the extent of IAA production. FIG. 4D shows the extent of IE production.

FIGS. 5A-5D show production of indole-containing compounds by bacterial strains of Bacillus licheniformis, Bacillus pumilus, and Weizmannia coagulans in addition to the Bacillus velezensis and Bacillus subtilis strains shown in FIGS. 4A-4D. FIG. 5A shows the extent of ILA production. FIG. 5B shows the extent of IAld production. FIG. 5C shows the extent of IAA production. FIG. 5D shows the extent of tryptophol (indole-3-ethanol, IE) production.

### DETAILED DESCRIPTION

Aspects of the present disclosure relate to compositions and nutritional supplements comprising microbial mixtures including one or more bacterial strains that produce one or more indole-containing molecules in the presence of L-tryptophan, and methods of use and production thereof. Without wishing to be bound by any particular theory, indole-containing molecules produced by metabolism of L-tryptophan are thought to act as agonists for the aryl hydrocarbon receptor (AhR). Ligand-binding to AhR facilitates a conformational change exposing the nuclear localization signal, causing translocation to the nucleus, and dimerization of ligand-bound AhR with AHR-nuclear translocator (ARNT) to produce a transcription factor complex that causes chromatin remodeling and transcription of target genes, including IL6, IL22, vascular endothelial growth factor A (VEGFA), cytochrome P450 1A1 (CYP1A1), cytochrome P450 1B1 (CYP1B1), and prostaglandin G/H synthase 2 (PTGS2). See, e.g., Hubbard et al., Drug Metab Dispos. (2015) 43(10):1522-1535. Indole-containing molecules (e.g., indole-3-carbinol, or indole) have been shown to inhibit proinflammatory responses in vivo and increase barrier function in intestinal epithelial cells.

Without wishing to be bound by any particular theory, the bacterial strains and combinations thereof described herein produce indole-containing metabolites in the gastrointestinal tract following administration to a subject, e.g., an animal or avian subject. The indole-containing metabolites are expected to stimulate AhR, thereby improving intestinal homeostasis (e.g., by modulating inflammation, improving intestinal epithelial cell barrier integrity). Production of such indole-containing molecules may be useful for treating, preventing, and/or alleviating symptoms associated with immune dysregulation, such as inflammation, allergy, diarrhea, itching, paw licking, head shaking, or vomiting, in a subject.

Generally observed behaviors in dogs such as scratching, incessant licking of paws, and shaking of the head can be indicative of underlying health conditions. The medical term for itchiness in dogs is pruritis, which can be brought on by various factors, including allergies to food, contact with environmental allergens such as dust mites or pollen, infestation of parasites like fleas or mites, and skin infections. Paw licking to a severe extent, commonly seen in conjunction with pruritis, could result in skin inflammation and irritation, worsening the original itchiness. Likewise, dogs shaking their heads may be signaling car infections, allergies or foreign objects lodged in their cars, causing discomfort and possibly leading to other issues if not addressed promptly. Addressing these symptoms is crucial for the dog's overall health and comfort. Currently recommended strategies for managing pruritic conditions usually involves a mix of measures, such as avoiding allergens, specialized washing, administration of anti-inflammatory drugs, and allergen-specific immunotherapy.

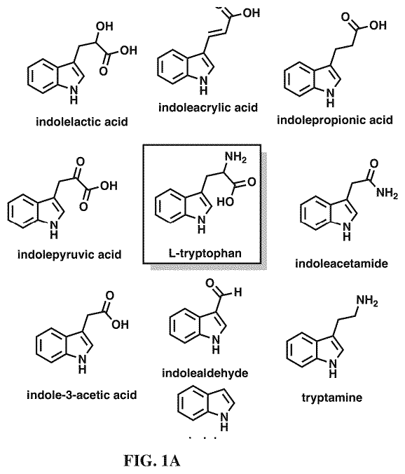

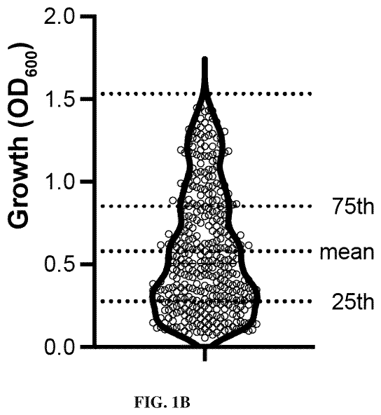

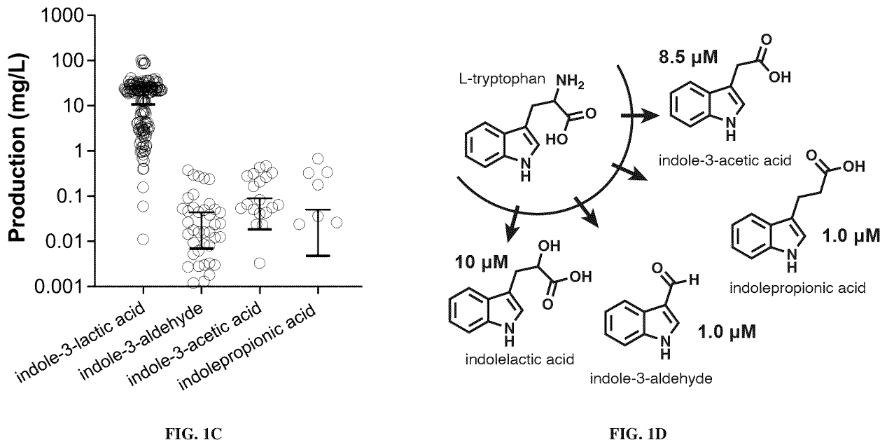

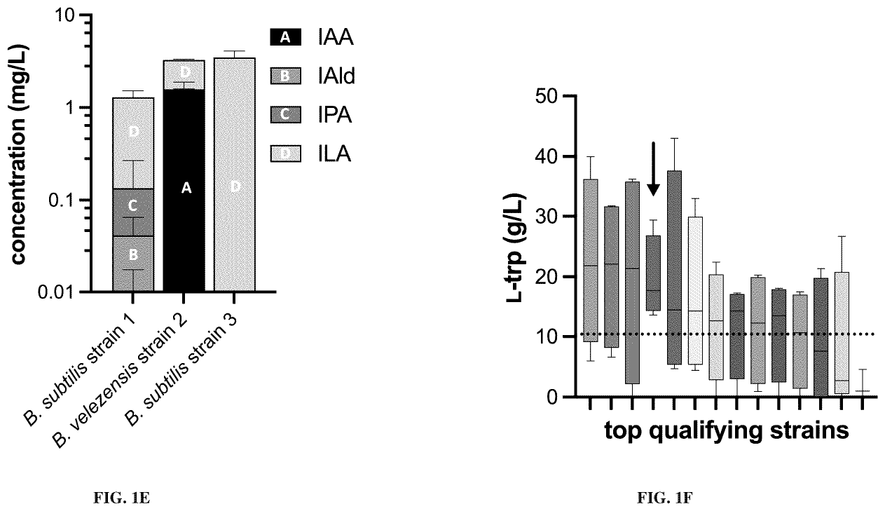

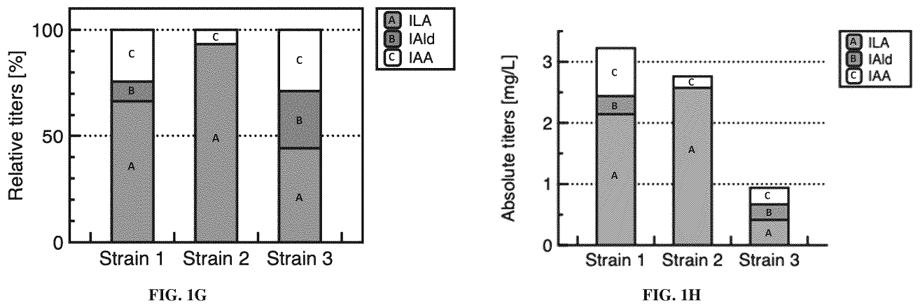

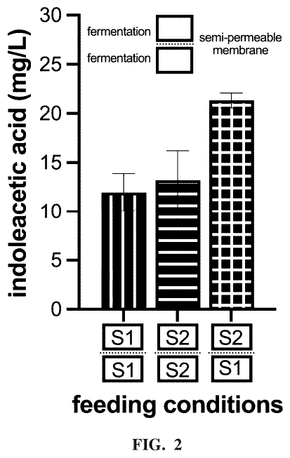

Consumption of beneficial microorganisms, namely bacteria, has been practiced for centuries due to the prevalence of fermented foods and beverages, e.g., yogurt, kimchi, beer. The consumed microorganisms can colonize or engraft anatomical niches (e.g., skin, gastrointestinal tract, lungs) of the host and propagate. Currently, purposeful colonization with beneficial microbes is being pursued as a clinical tool in human health as evident by the recent U.S. Food and Drug Administration (FDA) approval of a fecal microbial transplant. Temporally, this research dovetails with the desire of humans to improve the quality of life of their companion animals, namely dogs and cats. Currently, significant challenges for a canine-focused probiotic with validated immunoregulatory function include the rational design of the microbial communities and successful delivery of the product to the canine subject. Canonically, most canine probiotics are formulated with bacterial strains belonging to the genera Lactobacillus, Bifidobacterium, and Enterococcus. The inclusion of these microbes is based on non-specific qualifications of these microbes as traditional, safe, beneficial microbes. For canine supplements intended to ameliorate symptoms related to the immune dysregulation (e.g., inflammation), microbes capable of inducing desired immune responses are required. Moreso, products (e.g., nutritional supplements) containing these microbes should be formulated such that the desired phenotype (e.g., production of indole-containing molecules) can be carried out at the intended anatomical site (e.g., the canine intestinal mucosa). Unlike previous uses of single strains of Bacillus species to ameliorate disease (see, e.g., Bastos et al., BMC Vet Res. 2020. 16(1): 116; Mikawa et al., Open Vet J. 2021. 11(3):394-400; Schauf et al., J Appl Anim Nutr. 2019. 7(e3): 1-8; Acuff and Aldrich, J Anim Sci. 2021. 99(5):skab137; Vazquez-Mendoza et al., Microb Pathog. 2018. 114:458-463), the compositions and nutritional supplements described herein are prepared by combining bacterial strains to achieve strain-specific synergy based on metabolomic profiling, with this mechanism-based design allowing synergistic production, in increased amounts, of indole-containing molecules in the presence of L-tryptophan. FIG. 2 shows synergistic production of indoleacetic acid (IAA) when strains of B. subtilis and B. velezensis are grown adjacently separated by a permeable membrane that permits diffusion of small molecules and macromolecules. Additionally, metabolic profiling demonstrated that two strains of the same species (B. subtilis) could be beneficial, a strategy not taken in previous canine trials (FIGS. 1E-F). Specifically, this was driven by the findings that one B. subtilis strain is capable of protein hydrolysis to generate the free amino acid L-tryptophan, while the other produces a unique set of AHR agonists including a single molecule, indolepropionic acid, not produced by other strains in the community.

Stability, as measured by viability and function of the microbes over time and following administration, is a substantial concern, particularly in view of the exposure to stomach acid (pH 1-3) and degradation by proteases and bile acids. In the context of human therapeutics and prophylactics, microbes are generally not able to withstand these conditions without encapsulation or formulation with additional reagents-resulting in a higher cost that may prohibit widespread adaptation for animal subjects. Administration is also a specific problem for animal subjects, as formulation in a sensorially-attractive form is desired to aid or entice consumption. However, frequently an increased sensory experience requires addition of moisture (e.g., wet-food vs. dry kibble), as measured by water activity (a_{w}). As a_{w} increases, the survival of microbial strains decreases in the absence of expensive encapsulation techniques.

Also described herein are compositions, for example in the form of a dried powder, containing a rationally designed mixture of bacterial strains assembled to induce anti-inflammatory responses within the gastrointestinal tract of an animal subject (e.g., a canine subject) and be stable under a variety of manufacturing, storage, and delivery conditions, such as heat, high a_{w}, pH, osmotic stress over extended periods of time. This community of strains is uniquely able to facilitate anti-inflammatory responses by performing complimentary but distinct cross-feeding metabolisms to generate small molecules that are known to agonize the aryl-hydrocarbon receptor (AHR), an upstream sensor expressed across mammals that controls immune responses and has been shown to be an effective immunoregulatory system to control inflammation in murine and human biology. Furthermore, these strains were selected for their ability to withstand a variety of formulation strategies, storage temperatures, water activity levels, and administration methods including intake through the upper gastrointestinal tract.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### Bacterial Strains

Aspects of the present disclosure relate to compositions and nutritional supplements comprising at least three bacterial strains (e.g., 3, 4, 5, or more) having 16S rDNA sequences with at least 97% sequence identity to the nucleic acid sequences of SEQ ID NOs: 1-3. Aspects of the present disclosure relate to compositions and nutritional supplements comprising at least two bacterial strains (e.g., 2, 3, 4, 5, or more) wherein at least two bacterial strains have 16S rDNA sequences with at least 97% sequence identity to any of the nucleic acid sequences of SEQ ID NOs: 1-3. For example, the bacterial mixture of a composition or nutritional supplement may include a first bacterial strain having a 16S rDNA sequence with at least 97% sequence identity to the nucleic acid sequence of SEQ ID NO: 1, a second bacterial strain having a 16S rDNA sequence with at least 97% sequence identity to the nucleic acid sequence of SEQ ID NO: 2, and a third bacterial strain having a 16S rDNA sequence with at least 97% sequence identity to the nucleic acid sequence of SEQ ID NO: 3, wherein the first bacterial strain and the third bacterial strain are different bacterial strains. The skilled artisan will appreciate that different bacterial strains belonging to the same species (e.g., Bacillus subtilis) may comprise different 16S rDNA sequences and differ in one or more features, such as colony morphology. For example, in some embodiments, one bacterial strain comprising a 16S rDNA sequence with at least 97% sequence identity to SEQ ID NO: 1 has a flat colony morphology, and a second strain comprising a 16S rDNA sequence with at least 97% sequence identity to SEQ ID NO: 3 has a raised colony morphology. In some embodiments, a first bacterial strain comprising a 16S rDNA sequence with at least 97% sequence identity to SEQ ID NO: 1 has a rough surface colony morphology, and a second strain comprising a 16S rDNA sequence with at least 97% sequence identity to SEQ ID NO: 3 has a smooth surface colony morphology. In some embodiments, a first bacterial strain comprising a 16S rDNA sequence with at least 97% sequence identity to SEQ ID NO: 1 has a flat and rough colony morphology, and a second bacterial strain comprising a 16S rDNA sequence with at least 97% sequence identity to SEQ ID NO: 3 has a raised and smooth colony morphology. Such differences in colony morphology are routinely used in the art to distinguish bacterial strains In some embodiments, the bacterial mixture consists of a first bacterial strain having a 16S rDNA sequence with at least 97% sequence identity to the nucleic acid sequence of SEQ ID NO: 1, a second bacterial strain having a 16S rDNA sequence with at least 97% sequence identity to the nucleic acid sequence of SEQ ID NO: 2, and a third bacterial strain having a 16S rDNA sequence with at least 97% sequence identity to the nucleic acid sequence of SEQ ID NO: 3, wherein the first bacterial strain and the third bacterial strain are different bacterial strains.

In some embodiments, the first bacterial strain has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 1. In some embodiments, the second bacterial strain has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 2. In some embodiments, the third bacterial strain has a 16S rDNA sequence with at least 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% identity to SEQ ID NO: 3.

The bacterial strain comprising a 16S rDNA sequence provided by SEQ ID NO: 1 (also referred to herein as “Strain 1”) has the highest homology with a bacterial strain of the species Bacillus subtilis (RefSeq Assembly No. GCF_014805495.1):

Strain 1
16S ribosomal RNA coding sequence (16S rDNA)
(SEQ ID NO: 1)
AGGACGAACGCTGGCGGCGTGCCTAATACATGCAAGTCGAGCGGA
CAGATGGGAGCTTGCTCCCTGATGTTAGCGGCGGACGGGTGAGTA
ACACGTGGGTAACCTGCCTGTAAGACTGGGATAACTCCGGGAAAC
CGGGGCTAATACCGGATGGTTGTTTGAACCGCATGGTTCAAACAT
AAAAGGTGGCTTCGGCTACCACTTACAGATGGACCCGCGGCGCAT
TAGCTAGTTGGTGAGGTAACGGCTCACCAAGGCAACGATGCGTAG
CCGACCTGAGAGGGTGATCGGCCACACTGGGACTGAGACACGGCC
CAGACTCCTACGGGAGGCAGCAGTAGGGAATCTTCCGCAATGGAC
GAAAGTCTGACGGAGCAACGCCGCGTGAGTGATGAAGGTTTTCGG
ATCGTAAAGCTCTGTTGTTAGGGAAGAACAAGTACCGTTCGAATA
GGGCGGTACCTTGACGGTACCTAACCAGAAAGCCACGGCTAACTA
CGTGCCAGCAGCCGCGGTAATACGTAGGTGGCAAGCGTTGTCCGG
AATTATTGGGCGTAAAGGGCTCGCAGGCGGTTTCTTAAGTCTGAT
GTGAAAGCCCCCGGCTCAACCGGGGAGGGTCATTGGAAACTGGGG
AACTTGAGTGCAGAAGAGGAGAGTGGAATTCCACGTGTAGCGGTG
AAATGCGTAGAGATGTGGAGGAACACCAGTGGCGAAGGCGACTCT
CTGGTCTGTAACTGACGCTGAGGAGCGAAAGCGTGGGGAGCGAAC
AGGATTAGATACCCTGGTAGTCCACGCCGTAAACGATGAGTGCTA
AGTGTTAGGGGGTTTCCGCCCCTTAGTGCTGCAGCTAACGCATTA
AGCACTCCGCCTGGGGAGTACGGTCGCAAGACTGAAACTCAAAGG
AATTGACGGGGGCCCGCACAAGCGGTGGAGCATGTGGTTTAATTC
GAAGCAACGCGAAGAACCTTACCAGGTCTTGACATCCTCTGACAA
TCCTAGAGATAGGACGTCCCCTTCGGGGGCAGAGTGACAGGTGGT
GCATGGTTGTCGTCAGCTCGTGTCGTGAGATGTTGGGTTAAGTCC
CGCAACGAGCGCAACCCTTGATCTTAGTTGCCAGCATTCAGTTGG
GCACTCTAAGGTGACTGCCGGTGACAAACCGGAGGAAGGTGGGGA
TGACGTCAAATCATCATGCCCCTTATGACCTGGGCTACACACGTG
CTACAATGGACAGAACAAAGGGCAGCGAAACCGCGAGGTTAAGCC
AATCCCACAAATCTGTTCTCAGTTCGGATCGCAGTCTGCAACTCG
ACTGCGTGAAGCTGGAATCGCTAGTAATCGCGGATCAGCATGCCG
CGGTGAATACGTTCCCGGGCCTTGTACACACCGCCCGTCACACCA
CGAGAGTTTGTAACACCCGAAGTCGGTGAGGTAACCTTTTAGGAG
CCAGCCGCCGAAGGTGGGACAGATGATTGGGGTGAAGTCGTAACA
AGGTAGCCGTATCGGAAGGTGCGGCTGGATCACCTCCTTT

The bacterial strain comprising a 16S rDNA sequence provided by SEQ ID NO: 2 (also referred to herein as “Strain 2”) has the highest homology with a bacterial strain of the species Bacillus velezensis (RefSeq Assembly No. GCF_004115935.1):

Strain 2
16S ribosomal RNA coding sequence (16S rDNA)
(SEQ ID NO: 2)
AGACATAAAAGGTGGCTTCGGCTACCACTTACAGATGGACCCGCG
GCGCATTAGCTAGTTGGTGAGGTAACGGCTCACCAAGGCAACGAT
GCGTAGCCGACCTGAGAGGGTGATCGGCCACACTGGGACTGAGAC
ACGGCCCAGACTCCTACGGGAGGCAGCAGTAGGGAATCTTCCGCA
ATGGACGAAAGTCTGACGGAGCAACGCCGCGTGAGTGATGAAGGT
TTTCGGATCGTAAAGCTCTGTTGTTAGGGAAGAACAAGTGCCGTT
CAAATAGGGCGGCACCTTGACGGTACCTAACCAGAAAGCCACGGC
TAACTACGTGCCAGCAGCCGCGGTAATACGTAGGTGGCAAGCGTT
GTCCGGAATTATTGGGCGTAAAGGGCTCGCAGGCGGTTTCTTAAG
TCTGATGTGAAAGCCCCCGGCTCAACCGGGGAGGGTCATTGGAAA
CTGGGGAACTTGAGTGCAGAAGAGGAGAGTGGAATTCCACGTGTA
GCGGTGAAATGCGTAGAGATGTGGAGGAACACCAGTGGCGAAGGC
GACTCTCTGGTCTGTAACTGACGCTGAGGAGCGAAAGCGTGGGGA
GCGAACAGGATTAGATACCCTGGTAGTCCACGCCGTAAACGATGA
GTGCTAAGTGTTAGGGGGTTTCCGCCCCTTAGTGCTGCAGCTAAC
GCATTAAGCACTCCGCCTGGGGAGTACGGTCGCAAGACTGAAACT
CAAAGGAATTGACGGGGGCCCGCACAAGCGGTGGAGCATGTGGTT
TAATTCGAAGCAACGCGAAGAACCTTACCAGGTCTTGACATCCTC
TGACAATCCTAGAGATAGGACGTCCCCTTCGGGGGCAGAGTGACA
GGTGGTGCATGGTTGTCGTCAGCTCGTGTCGTGAGATGTTGGGTT
AAGTCCCGCAACGAGCGCAACCCTTGATCTTAGTTGCCAGCATTC
AGTTGGGCACTCTAAGGTGACTGCCGGTGACAAACCGGAGGAAGG
TGGGGATGACGTCAAATCATCATGCCCCTTATGACCTGGGCTACA
CACGTGCTACAATGGACAGAACAAAGGGCAGCGAAACCGCGAGGT
TAAGCCAATCCCACAAATCTGTTCTCAGTTCGGATCGCAGTCTGC
AACTCGACTGCGTGAAGCTGGAATCGCTAGTAATCGCGGATCAGC
ATGCCGCGGTGAATACGTTCCCGGGCCTTGTACACACCGCCCGTC
ACACCACGAGAGTTTGTAACACCCGAAGTCGGTGAGGTAACCTTT
TAGGAGCCAGCCGCCGAAGGTGGGACAGATGATTGGGGTGAAGTC
GTAACAAGGTAGCCGTATCGGAAGGTGCGGCTGGATCACCTCCTT
T

The bacterial strain comprising a 16S rDNA sequence provided by SEQ ID NO: 3 (also referred to herein as “Strain 3”) has the highest homology with a bacterial strain of the species Bacillus subtilis (RefSeq Assembly No. GCF_003046665.1):

Strain 3
16S ribosomal RNA coding sequence (16S rDNA)
(SEQ ID NO: 3)
CGCTGGCGGCGTGCCTAATACATGCAAGTCGAGCGGACAGATGGG
AGCTTGCTCCCTGATGTTAGCGGCGGACGGGTGAGTAACACGTGG
GTAACCTGCCTGTAAGACTGGGATAACTCCGGGAAACCGGGGCTA
ATACCGGATGGTTGTTTGAACCGCATGGTTCAAACATAAAAGGTG
GCTTCGGCTACCACTTACAGATGGACCCGCGGCGCATTAGCTAGT
TGGTGAGGTAACGGCTCACCAAGGCAACGATGCGTAGCCGACCTG
AGAGGGTGATCGGCCACACTGGGACTGAGACACGGCCCAGACTCC
TACGGGAGGCAGCAGTAGGGAATCTTCCGCAATGGACGAAAGTCT
GACGGAGCAACGCCGCGTGAGTGATGAAGGTTTTCGGATCGTAAA
GCTCTGTTGTTAGGGAAGAACAAGTACCGTTCGAATAGGGCGGTA
CCTTGACGGTACCTAACCAGAAAGCCACGGCTAACTACGTGCCAG
CAGCCGCGGTAATACGTAGGTGGCAAGCGTTGTCCGGAATTATTG
GGCGTAAAGGGCTCGCAGGCGGTTTCTTAAGTCTGATGTGAAAGC
CCCCGGCTCAACCGGGGAGGGTCATTGGAAACTGGGGAACTTGAG
TGCAGAAGAGGAGAGTGGAATTCCACGTGTAGCGGTGAAATGCGT
AGAGATGTGGAGGAACACCAGTGGCGAAGGCGACTCTCTGGTCTG
TAACTGACGCTGAGGAGCGAAAGCGTGGGGAGCGAACAGGATTAG
ATACCCTGGTAGTCCACGCCGTAAACGATGAGTGCTAAGTGTTAG
GGGGTTTCCGCCCCTTAGTGCTGCAGCTAACGCATTAAGCACTCC
GCCTGGGGAGTACGGTCGCAAGACTGAAACTCAAAGGAATTGACG
GGGGCCCGCACAAGCGGTGGAGCATGTGGTTTAATTCGAAGCAAC
GCGAAGAACCTTACCAGGTCTTGACATCCTCTGACAATCCTAGAG
ATAGGACGTCCCCTTCGGGGGCAGAGTGACAGGTGGTGCATGGTT
GTCGTCAGCTCGTGTCGTGAGATGTTGGGTTAAGTCCCGCAACGA
GCGCAACCCTTGATCTTAGTTGCCAGCATTCAGTTGGGCACTCTA
AGGTGACTGCCGGTGACAAACCGGAGGAAGGTGGGGATGACGTCA
AATCATCATGCCCCTTATGACCTGGGCTACACACGTGCTACAATG
GACAGAACAAAGGGCAGCGAAACCGCGAGGTTAAGCCAATCCCAC
AAATCTGTTCTCAGTTCGGATCGCAGTCTGCAACTCGACTGCGTG
AAGCTGGAATCGCTAGTAATCGCGGATCAGCATGCCGCGGTGAAT
ACGTTCCCGGGCCTTGTACACACCGCCCGTCACACCACGAGAGTT
TGTAACACCCGAAGTCGGTGAGGTAACCTTTTAGGAGCCAGCCGC
CGAAGGTGGGACAGATGATTGGGGTGAAGTCGTAACAAGGTAGCC
GTATCGGAAGGTGCGGCTGGATCACCTCCTTT

Strain 1 has a V4 region (a region of the 16S rDNA locus known in the art as useful for identification) according to the nucleic acid sequence of SEQ ID NO: 4:

Strain 1
16S rDNA V4 region
(SEQ ID NO: 4)
GTGCCAGCAGCCGCGGTAATACGTAGGTGGCAAGCGTTGTCCGGA
ATTATTGGGCGTAAAGGGCTCGCAGGCGGTTTCTTAAGTCTGATG
TGAAAGCCCCCGGCTCAACCGGGGAGGGTCATTGGAAACTGGGGA
ACTTGAGTGCAGAAGAGGAGAGTGGAATTCCACGTGTAGCGGTGA
AATGCGTAGAGATGTGGAGGAACACCAGTGGCGAAGGCGACTCTC
TGGTCTGTAACTGACGCTGAGGAGCGAAAGCGTGGGGAGCGAACA
GGATTAGA

Strain 2 has a V4 region according to the nucleic acid sequence of SEQ ID NO: 5:

Strain 2
16S rDNA V4 region
(SEQ ID NO: 5)
GTGCCAGCAGCCGCGGTAATACGTAGGTGGCAAGCGTTGTCCGGA
ATTATTGGGCGTAAAGGGCTCGCAGGCGGTTTCTTAAGTCTGATG
TGAAAGCCCCCGGCTCAACCGGGGAGGGTCATTGGAAACTGGGGA
ACTTGAGTGCAGAAGAGGAGAGTGGAATTCCACGTGTAGCGGTGA
AATGCGTAGAGATGTGGAGGAACACCAGTGGCGAAGGCGACTCTC
TGGTCTGTAACTGACGCTGAGGAGCGAAAGCGTGGGGAGCGAACA
GGATTAGA

Strain 3 has a V4 region according to the nucleic acid sequence of SEQ ID NO: 6:

Strain 3
16S rDNA V4 region
(SEQ ID NO: 6)
GTGCCAGCAGCCGCGGTAATACGTAGGTGGCAAGCGTTGTCCGGA
ATTATTGGGCGTAAAGGGCTCGCAGGCGGTTTCTTAAGTCTGATG
TGAAAGCCCCCGGCTCAACCGGGGAGGGTCATTGGAAACTGGGGA
ACTTGAGTGCAGAAGAGGAGAGTGGAATTCCACGTGTAGCGGTGA
AATGCGTAGAGATGTGGAGGAACACCAGTGGCGAAGGCGACTCTC
TGGTCTGTAACTGACGCTGAGGAGCGAAAGCGTGGGGAGCGAACA
GGATTAGA

In some embodiments, the bacterial mixture comprises two or more bacterial strains belonging to the species Bacillus subtilis, and one or more bacterial strains belonging to the species Bacillus velezensis, wherein the bacterial strains belonging to the species Bacillus subtilis are different bacterial strains. For example, in some embodiments, one bacterial strain of Bacillus subtilis has a flat colony morphology, and a second strain of Bacillus subtilis has a raised colony morphology. In some embodiments, a first bacterial strain of Bacillus subtilis has a rough surface colony morphology, and a second strain of Bacillus subtilis has a smooth surface colony morphology. In some embodiments, a first bacterial strain of Bacillus subtilis has a flat and rough colony morphology, and a second bacterial strain of Bacillus subtilis has a raised and smooth colony morphology. Such differences in colony morphology are routinely used in the art to distinguish bacterial strains. In some embodiments, the bacterial mixture comprises two bacterial strains belonging to Bacillus subtilis. In some embodiments, the bacterial mixture comprises one bacterial strain belonging to Bacillus velezensis. In some embodiments, the bacterial mixture comprises 2, 3, 4, 5, 6, 7, 8, 9, or 10 bacterial strains belonging to Bacillus subtilis. In some embodiments, the bacterial mixture comprises 1, 2, 3, 4, 5, 6, 7, 8, 9, or 10 bacterial strains belonging to Bacillus velezensis. In some embodiments, the bacterial mixture consists of a first bacterial strain belonging to Bacillus subtilis, a second bacterial strain belonging to Bacillus velezensis, and a third bacterial strain belonging to Bacillus subtilis. In some embodiments, the first bacterial strain belonging to Bacillus subtilis comprises a 16S rDNA sequence with at least 97% sequence identity to the nucleic acid sequence of SEQ ID NO: 1, the third bacterial strain belonging to Bacillus subtilis comprises a 16S rDNA sequence with at least 97% sequence identity to the nucleic acid sequence of SEQ ID NO: 3, and the 16S rDNA sequences of the first bacterial strain and the third bacterial strains are not 100% identical. In some embodiments, the 16S rDNA sequence of the first bacterial strain belonging to Bacillus subtilis has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to 16S rDNA sequence of the third bacterial strain belonging to Bacillus subtilis.

Bacterial mixtures of the compositions or nutritional supplements described herein may include one or more additional bacterial strains. Additional bacterial strains may be taxonomically or phylogenetically related to bacterial strains of Bacillus subtilis or Bacillus velezensis described herein. For example, in some embodiments, one or more of the additional bacterial strains belongs to the same phylum, class, order, family, genus, or species as another strain described herein. In some embodiments, one or more additional bacterial strains belong to the phylum Bacillota. In some embodiments, one or more additional bacterial strains belong to the class Bacilli. In some embodiments, one or more additional bacterial strains belong to the family Bacillaceae. In some embodiments, one or more additional bacterial strains belong to the genus Bacillus. In some embodiments, one or more additional bacterial strains belong to the species Bacillus subtilis. In some embodiments, one or more additional bacterial strains belong to the species Bacillus velezensis.

In some embodiments, a bacterial mixture comprises 2, 3, 4, 5, 6, 7, 8, 9, 10, 11, 12, 13, 14, 15, 16, 17, 18, 19, or 20 additional bacterial strains belonging to the genus Bacillus. In some embodiments, a bacterial mixture comprises 2, 3, 4, 5, 6, 7, 8, 9, 10, 11, 12, 13, 14, 15, 16, 17, 18, 19, or 20 additional bacterial strains belonging to the species Bacillus subtilis. In some embodiments, a bacterial mixture comprises 2, 3, 4, 5, 6, 7, 8, 9, 10, 11, 12, 13, 14, 15, 16, 17, 18, 19, or 20 additional bacterial strains belonging to the species Bacillus velezensis. In some embodiments, the 16S rDNA sequence of an additional bacterial strain belonging to Bacillus subtilis is not identical to SEQ ID NO: 1 or SEQ ID NO: 3. In some embodiments, an additional bacterial strain belonging to Bacillus subtilis comprises a 16S rDNA sequence that has at least 97%, but less than 100%, sequence identity to the nucleic acid sequence of SEQ ID NO: 1. In some embodiments, an additional bacterial strain belonging to Bacillus subtilis comprises a 16S rDNA sequence that has at least 97%, but less than 100%, sequence identity to the nucleic acid sequence of SEQ ID NO: 3. In some embodiments, the 16S rDNA sequence of an additional bacterial strain belonging to Bacillus velezensis is not identical to SEQ ID NO: 2. In some embodiments, an additional bacterial strain belonging to Bacillus velezensis comprises a 16S rDNA sequence that has at least 97%, but less than 100%, sequence identity to the nucleic acid sequence of SEQ ID NO: 2.

In some embodiments, any of the bacterial mixtures described herein further comprises one or more additional spore-forming strains belonging to the genus Bacillus. In some embodiments, the bacterial mixture further comprises a bacterial strain belonging to the species Bacillus amyloliquefaciens. In some embodiments, the bacterial mixture further comprises a bacterial strain belonging to the species Bacillus lentus. In some embodiments, the bacterial mixture further comprises a bacterial strain belonging to the species Bacillus licheniformis. In some embodiments, the bacterial mixture further comprises a bacterial strain belonging to the species Bacillus pumilus.

In some embodiments, the 16S rDNA sequence of an additional bacterial strain belonging to Bacillus subtilis has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to SEQ ID NO: 1. In some embodiments, the 16S rDNA sequence of an additional bacterial strain belonging to Bacillus subtilis has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to SEQ ID NO: 3. In some embodiments, the 16S rDNA sequence of an additional bacterial strain belonging to Bacillus velezensis has no more than 99.9%, 99.8%, 99.7%, 99.6%, 99.5%, 99.4%, 99.3%, 99.2%, 99.1%, 99.0%, 98.8%, 98.6%, 98.5%, 98.4%, 98.2%, 98.0%, 97.8%, 97.6%, 97.5%, 97.4%, or 97.2% identity to SEQ ID NO: 2.

The compositions and nutritional supplements described herein comprise bacterial mixtures containing three or more bacterial strains. In some embodiments, the compositions described herein comprise at least 4, at least 5, at least 6, at least 7, at least 8, at least 9, at least 10, at least 11, at least 12, at least 13, at least 14, at least 15, at least 16, at least 17, at least 18, at least 19, or up to 20 total bacterial strains. In some embodiments, the compositions or nutritional supplements described herein comprise 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 bacterial strains. In some embodiments, the compositions or nutritional supplements comprise a mixture of bacterial strains that consists of 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 strains.

It will be appreciated that the terms “bacterial strains,” “microbial strains” “microbes,” “bacterial cells” and “microorganisms” are used interchangeably herein.

Bacterial strains identified as described herein were compared with sequences in publicly available nucleic acid databases, such as Basic Local Alignment Search Tool (BLAST) to determine closely related genera and species and were analyzed using taxonomic assignment tools, such as RDP Classifier, which assign bacterial taxonomy to representative sequences.

Ribosomal 16S DNA sequences are provided below for representative bacterial strains. The closest related identified bacterial species were determined based on whole genome sequence analysis and a combination of analysis of full length 16S rDNA sequences and the sequences of single-copy conserved genes compared to publicly available sequence databases. It should be appreciated that multiple bacterial strains disclosed herein may have the highest homology with the same species. Bacterial strains having the same or highly related 16S rDNA variable region sequences (and/or belonging to the same species) may be phenotypically distinct. For example, two different bacterial strains may differ phenotypically by growth rate, antibiotic resistance, phage resistance, cell wall thickness, motility, competence, spore-forming ability, rates of metabolite (e.g., indolepropionic acid) production, protein degradation and/or amino acid (e.g., L-tryptophan) production, ability to produce a given metabolite, and/or ability to degrade a certain molecule. It should further be appreciated that the bacterial strains disclosed herein that have a 16S rDNA sequence with a nucleic acid sequence selected from the group consisting of SEQ ID NOs: 1-3, are also homologous to other strains based on their whole genome sequence, or subset of their whole genome sequence.

It should further be appreciated that the bacterial species described herein may be identified based on the nucleotide sequence of the full length 16S rDNA. Alternatively or in addition, the bacterial species described herein may be identified based on identification of 16S sequences through whole genome sequencing, and by comparing the sequences with 16S databases, or comparing the whole genome sequence, or a subset of their whole genome sequence to sequence databases.

It should be appreciated that the compositions may include multiple strains of a particular species. In some embodiments, the composition includes multiple strains of a particular species that are obtained from independent sources but the strains have the same or highly related 16S rDNA sequences.

Aspects of the disclosure relate to bacterial strains with 16S rDNA sequences that have sequence identity to a nucleic acid sequence of any one of the sequences of the bacterial strains or bacterial species described herein. The terms “identical,” or percent “identity,” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over the entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the recited degree of identity exists over the length of the 16S rRNA or 16S rDNA sequence.

In some embodiments, the bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity relative to any of the bacterial strains or bacterial species described herein over a specified region (such as a region of the 16S rDNA sequences provided herein) or over the entire sequence (such as the entire 16S rDNA). It would be appreciated by one of skill in the art that the term “sequence identity” or “percent sequence identity,” in the context of two or more nucleic acid sequences or amino acid sequences, refers to a measure of similarity between two or more sequences or portion(s) thereof. In some embodiments, the identity exists over the length of the 16S rRNA or 16S rDNA sequence.

Additionally, or alternatively, two or more sequences may be assessed for the alignment between the sequences. An alignment of 100% or “total alignment” referring to two or more nucleic acids or amino acid sequences, refers to two or more sequences or subsequences that are the same. Two sequences are “substantially aligned” if two sequences have less than 100% alignment, such as a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length.

For sequence comparison, typically one sequence acts as a reference sequence, to which test sequences are compared. Methods of alignment of sequences for comparison, including measuring identity between sequences, are well known in the art. See, e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1988) 85:2444, by computerized implementations of these algorithms (GAP, BESTFIT, FASTA, and TFASTA in the Wisconsin Genetics Software Package, Genetics Computer Group. Madison, WI), or by manual alignment and visual inspection (see. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. (1977) 25:3389-3402; and Altschul et al., J. Mol. Biol. (1990) 215:403-410, respectively.

As will be appreciated by one of ordinary skill in the art, nomenclature regarding bacterial genus and species names may be reclassified to reflect phylogenetic relationships of microorganisms. See, e.g., Zheng et al. Inter. J. System. And Evol. Microbiol. (2020) 70(4).

### Nutritional Supplements

Some aspects of the disclosure relate to nutritional supplements comprising any of the compositions or bacterial mixtures described herein. Also within the scope of the disclosure are food products comprising any of the bacterial strains described herein and a nutrient. Food products and nutritional supplements are, in general, intended for the consumption of a human or an animal, such as a companion animal (e.g., a dog) or a bird. Any of the compositions or microbial mixtures described herein may be present in a food product or nutritional supplement. In some embodiments, the bacterial strains are present in spore form in a nutritional supplement. In some embodiments, the bacterial strains are present in vegetative form in a nutritional supplement. In some embodiments, the nutritional supplement comprises both vegetative bacteria and bacteria in spore form. The compositions disclosed herein can be used in a food or beverage, such as a health food, a pet food, a functional food, a dietary supplement, a food or beverage for patients, or an animal feed.

Non-limiting examples of the forms of nutritional supplements include carbohydrate-containing foods such as rice food products; paste products such as fish hams, sausages, paste products of seafood; pouch products. Further, the examples also include food products and nutritional supplements prepared in the forms of powders, granules, kibbles, tablets, capsules, liquids, pastes, chews, and jellies.

Any suitable form may be used for a food product or nutritional supplement, for example for consumption by a subject (e.g., a mammalian subject, such as a dog or cat). Non-limiting examples of form factors include chews, such as chews with softer interiors than exteriors; kibbles; and spreads.

As will be appreciated by one of ordinary skill in the art, a food product provides a primary caloric and nutritive source intended for consumption by a subject (e.g., a feed or treat). In contrast, a nutritional supplement is not intended to provide a primary caloric or nutritive source for a subject but rather provides a targeted effect. The nutritional supplements described herein are capable of treat, preventing, and/or reducing a symptom associated with inflammation, diarrhea vomiting, itching, paw licking, head shaking, or skin irritation, and/or one or more allergy symptoms in a subject. Nutritional supplements may provide additional nutrients or functional ingredients that complement a standard dict, e.g., a standard pet diet.

In some embodiments, the nutritional supplement is a chew having a soft interior. In some embodiments, the nutritional supplement is a chew having a soft interior. In some embodiments, the nutritional supplement is a kibble. In some embodiments, the kibble is an extruded kibble. Extruded and/or injection-molded chews, sticks, and kibbles are known in the art, and any suitable method may be used to manufacture such chews, sticks, and kibbles. In some embodiments, the food product, or nutritional supplement is a soft jerky chew. Jerky chews may be made from any suitable starting material, such as dried meat. In some embodiments, the food product or nutritional supplement is a spread. In some embodiments, the spread comprises one or more nuts. In some embodiments, the spread is a peanut butter spread. The skilled artisan will appreciate that a food product or supplement intended for consumption by a particular animal will not contain ingredients that are toxic to that animal (e.g., macadamia nuts for dogs).

In some embodiments, the nutritional supplement is an additive. Additives may be added to other foods or food products before consumption by an animal. In some embodiments, the nutritional supplement is a water additive. Water additives are added to drinking water. In some embodiments, the nutritional supplement is a meal topper. Meal toppers may be added directly to food before consumption, with or without incorporation before consumption. In some embodiments, the nutritional supplement is a liquid spray. Liquid sprays may be administered directly to an animal, or sprayed on food or in water, with or without incorporation before consumption.

In some embodiments, the nutritional supplement comprises any of the bacterial mixtures as described herein, and a carrier. The carrier may contain any combination of nutrients, excipients, diluents, and/or binders. In some embodiments, the carrier comprises one or more of the following compounds: water, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, oil (e.g., coconut oil) glycerin (e.g., coconut glycerin), glucose, sucrose, sorbitol, lactose, whey, whey permeate, wheat flour, wheat bran, corn gluten meal, starch and cellulose. In some embodiments, the carrier is a gelatinized starch matrix. In some embodiments, the carrier is maltodextrin. In some embodiments, the carrier is a plant-based maltodextrin. In some embodiments, the carrier is tapioca maltodextrin. In some embodiments, the carrier is corn maltodextrin. Any suitable method may be used to obtain maltodextrin from a plant, such as isolation from one or more parts of the plant.

Nutritional supplements containing the bacterial strains described herein may be produced using methods known in the art and may contain the same amount or number of bacteria (e.g., by weight, amount, or CFU) as the compositions described herein. Selection of an appropriate amount or number of bacteria in the food product may depend on various factors, including for example, the serving size of the food product, the frequency of consumption of the food product, the specific bacterial strains contained in the nutritional supplement, the amount of water in the food product, and/or additional conditions for survival of the bacteria in the nutritional supplement. In some embodiments, a nutritional supplement comprises 10⁶ to 10¹² colony forming units (CFUs) of each bacterial strain of a microbial mixture, per gram of the nutritional supplement. In some embodiments, the nutritional supplement comprises 10⁸ to 10¹⁰ CFUs of each bacterial strain of the microbial mixture per gram of the nutritional supplement.

Nutritional supplements and/or food products may comprise one or more nutrients in addition to the bacterial strains. For example, a nutritional supplement and/or food product may comprise a carbohydrate or carbohydrate source. Non-limiting examples of carbohydrate sources include grains, such as corn, rice, milo, sorghum, barley, alfalfa, oats, and wheat. In some embodiments, the nutritional supplement comprises a grain. In some embodiments, the nutritional supplement comprises wheat, corn, rice, oats, and/or barley. The nutritional supplement may also comprise a gelatinized starch matrix. Matrices of gelatinized starch may comprise any starch listed herein, or another starch known in the art.

Nutritional supplements and/or food products may comprise a fiber. Fibers may add bulk to a composition, such as a nutritional supplement, and be fermented by intestinal bacteria to produce short-chain fatty acids and other metabolites. Non-limiting examples of fibers include beet pulp (e.g., sugar beet pulp), gum arabic, gum talha, psyllium, rice bran, carob bean gum, citrus pulp, pectin, fructooligosaccharide, mannaoligofructose, soy fiber, arabinogalactan, galactooligosaccharide, arabinoxylan, chicory pulp, chicory, coconut endosperm fiber, wheat fiber, and inulin.

A nutritional supplement and/or food product may comprise an algal strain. Algae may add one or more nutrients and/or bulk to a composition. Non-limiting examples of algal species include Ascophyllum nodosum, Spirulina (e.g., Arthrospira platensis, Arthrospira fusiformis, and Arthrospira maxima), and/or Fucus visculosus.

Nutritional supplements and/or food products may comprise one or more inorganic minerals. Non-limiting examples of minerals include Calcium carbonate, calcium, boron, selenium, calcium chloride, chloride, ferrous fumarate, zinc acetate, choline chloride, chromium, ferrous gluconate, zinc sulfate, chromium, tripicolinate, cobalt, magnesium oxide, zinc gluconate, dicalcium phosphate, copper, magnesium sulfate, ferrous sulfate, iodine, magnesium carbonate, monosodium phosphate, iron, chromium picolinate, potassium chloride, magnesium, calcium citrate, potassium citrate, manganese, calcium lactate, potassium sorbate, phosphorus, calcium gluconate, sodium bisulfate, potassium, chromium chloride, sodium hexametaphosphate, sodium, chromium nicotinate, tricalcium phosphate, zinc, and chromium citrate.

Nutritional supplements and/or food products may comprise one or more protein sources. Non-limiting examples of such protein sources include chicken meals, chicken, chicken byproduct meals, eggs, lamb, lamb meals, turkey, turkey meals, beef, beef by-products, pork, pork by-products, viscera, fish, fish meal, enterals, kangaroo, white fish, venison, blood marrow, bone marrow, chickpeas, chickpea meal, soybean meal, soy protein isolate, and soy protein concentrate.

Nutritional supplements and/or food products may comprise one or more starches. Non-limiting examples of such starches include cereals, grains, corn, wheat, rice, oats, corn grits, sorghum, grain sorghum, milo, wheat bran, oat bran, amaranth, durum, and semolina.

Nutritional supplements and/or food products may comprise one or more dairy products. Dairy products include, without limitation, cream, milk, butter, and cheese.

In some embodiments, any of the nutritional supplements described herein may comprise a meat or animal-derived material. In some embodiments, the meat or animal-derived material is beef, pork, chicken, eggs, turkey, lamb, fish, blood marrow, bone marrow, or a combination of any thereof.

Some aspects of the disclosure relate to methods of producing nutritional supplements by combining a bacterial mixture or composition as described herein with a carrier. Bacterial strains and other components of a nutritional supplement, such as a carrier, excipient, or diluent, may be combined in any amount suitable for producing a final mixture having one or more desired characteristics, such as volume, texture, bacterial amount, and/or nutritional content. Bacterial strains may be cultured independently and combined to produce a bacterial mixture, which is then combined with other components of a nutritional supplement. Alternatively, bacterial strains may be cultured together, and the community may be combined with other components of a composition or nutritional supplement. Nutritional supplements containing bacterial strains may be prepared by mixing bacterial strains with other ingredients to produce any solid or liquid formulation as described herein, such as rice food products; paste products such as fish hams, sausages, paste products of seafood; pouch products, powders, granules, kibbles, tablets, chews, capsules, liquids, pastes, and jellies.

Bacterial strains may be combined with a humectant in a nutritional supplement. Humectants preserve or retain moisture in a composition (e.g., a nutritional supplement), thereby preventing the composition from becoming undesirably dry or developing an undesirably brittle texture. In some embodiments, a humectant is included in a nutritional supplement to reduce water activity. In some embodiments, the humectant is glycol. In some embodiments, the bacterial strains are dried (e.g., spray-dried or lyophilized) before being combined with one or more other components of a nutritional supplement. In some embodiments, the bacterial strains are in spore form when they are combined with one or more other components of a nutritional supplement. In some embodiments, bacterial strains are combined with a gelatinized starch matrix or a starch matrix alternative. In some embodiments, bacterial strains are combined with a soluble or insoluble fiber. In some embodiments, the bacterial strains are combined with maltodextrin, sugar beet pulp, chicory pulp, chicory, coconut endosperm fiber, wheat fiber, and/or inulin . . . . In some embodiments, the bacterial strains are combined with corn maltodextrin. In some embodiments, the bacterial strains are combined with inulin.

### Methods of Use

Some aspects of the disclosure relate to administration of any of the compositions or nutritional supplements described herein to a subject in need thereof. Administration may be accomplished through any means known in the art. In some embodiments, any of the compositions or nutritional supplements described herein are administered orally. Oral administration may refer to manual administration, such as oral gavage or spraying (e.g., via aerosol). Oral administration may also refer to feeding, such as providing the composition or nutritional supplement to a subject (e.g., a companion animal), which ingests it by eating or drinking. Methods may include a single administration or administration may be repeated. Repeat administrations may be conducted at regular intervals (e.g., daily or weekly). For example, a subject may be administered a composition or supplement one or more times daily, over a specified time period, such as a week, several weeks, a month, or several months. Alternatively, multiple instances of administration may be conducted on an as-needed basis. In some embodiments, a composition or nutritional supplement is administered rectally. In some embodiments, the composition or supplement is in the form of a suppository when administered rectally. In some embodiments, bacterial strains of a composition or nutritional supplement are delivered to the intestine. In some embodiments, bacteria are delivered to the colon. Such delivery to the intestine or colon may be accomplished by oral or rectal administration.

In some embodiments, a composition or nutritional supplement is administered in an effective amount for treating, preventing, or alleviating one or more symptoms in a subject. An “effective amount” or “effective dosage” refers to an amount or dosage, respectively, sufficient to treat, alleviate, or prevent a sign or symptom in a subject. The actual effective amount or dosage will depend on the individual subject and their health. Such effective amounts or dosages may be determined by routine assays known to those of skill in the art. In some embodiments, a composition or nutritional supplement is administered in an effective amount to treat or prevent a symptom associated with inflammation. In some embodiments, the composition or nutritional supplement is administered in an effective amount to reduce inflammation in the subject. In some embodiments, the composition or nutritional supplement is administered in an effective amount to treat or prevent diarrhea in the subject. In some embodiments, the composition or nutritional supplement is administered in an effective amount to treat or prevent vomiting in the subject. In some embodiments, the composition or nutritional supplement is administered in an effective amount to treat or prevent itching, paw licking, head shaking, or skin irritation, in the subject. In some embodiments, the composition or nutritional supplement is administered in an effective amount to treat or prevent one or more allergy symptoms in the subject.

Subjects to which any of the compositions or nutritional supplements described herein may be administered include any animal known in the art. In some embodiments, the subject is a human. In some embodiments, the animal is a domesticated animal. In some embodiments, the animal is a carnivore. In some embodiments, the animal is a rodent. In some embodiments, the rodent is a mouse, rat, guinea pig, chinchilla, or hamster. In some embodiments, the animal is a dog, cat, rabbit, guinea pig, hamster, or ferret. In some embodiments, the animal is a dog. In some embodiments, the animal is a bovine, swine, llama, alpaca, sheep, or goat. In some embodiments, the animal is a bird.

In some embodiments, the compositions and nutritional supplements described herein are effective in treating or preventing one or more symptoms or adverse events in a subject. As discussed above, the bacterial mixtures are capable of producing indole-containing molecules such as those produced by metabolism of L-tryptophan act as agonists of the aryl hydrocarbon receptor (Ahr), thereby improving immune homeostasis. In some embodiments, the compositions and nutritional supplements are effective in reducing one or more symptoms, such as inflammation, diarrhea, vomiting, itching, paw licking, head shaking, or allergy. In some embodiments, the compositions and nutritional supplements reduce inflammation in the subject. In some embodiments, the compositions and nutritional supplements treat or prevent vomiting in the subject. In some embodiments, the compositions and nutritional supplements treat or prevent itching, paw licking, and/or head shaking in the subject. In some embodiments, the compositions and nutritional supplements treat or prevent allergy in the subject.

In some embodiments, the compositions and nutritional supplements reduce inflammation in the subject. In some embodiments, the compositions and nutritional supplements treat or prevent one or more symptoms associated with inflammation. Methods of measuring inflammation are known in the art, and include, without limitation, measuring the concentration of one or more cytokines (e.g., TNF-α, IL-1β, IL-6, IL-10, and IL-12) in the subject, such as in a sample obtained from the subject (e.g., a blood sample, serum sample, plasma sample).

In some embodiments, the compositions and nutritional supplements described herein may be effective in reducing one or more symptoms, such as inflammation, diarrhea, vomiting, itching, paw licking, head shaking, constipation, anal gland inflammation, lack of appetite, lethargy, arthritis, excessive grooming, abdominal pain, and fever. In some embodiments, the compositions and nutritional supplements treat or prevent diarrhea in the subject. In some embodiments, the compositions and nutritional supplements treat or prevent vomiting or nausea in the subject. In some embodiments, the compositions and nutritional supplements treat or prevent itching, paw licking, head shaking, and/or skin irritation in the subject. In some embodiments, the compositions or nutritional supplements treat or prevent one or more allergy symptoms in the subject. Non-limiting examples of allergy symptoms include nasal discharge, watery eyes, coughing, sneezing, itching, paw licking, head shaking, and rash.

The extent to which a composition or nutritional supplement treats or prevents a symptom may be evaluated using methods known in the art, such as monitoring the change in the frequency, occurrence, and/or severity of symptoms following administration of the composition or nutritional supplement. Additionally, groups of subjects administered the bacterial mixture, composition, or nutritional supplement may be monitored and compared to another group of subjects that was not administered the composition or nutritional supplement, a group that was administered a different amount of the composition or nutritional supplement, and/or a group that was administered a control composition (e.g., a carrier or excipient lacking the bacterial strains).

In some embodiments, the composition and/or nutritional supplement reduces the serum concentration of one or more inflammatory cytokines in the subject. In some embodiments, the serum cytokine concentration is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more as compared to the serum cytokine concentration in the same subject prior to prior to exposure to the composition or nutritional supplement. In some embodiments, the serum cytokine concentration is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the serum cytokine concentration in the same subject prior to exposure to the composition or nutritional supplement.

In some embodiments, the composition and/or nutritional supplement reduces the duration of inflammation in the subject. In some embodiments, the duration of inflammation is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the duration of inflammation in the same subject prior to exposure to the composition or nutritional supplement. In some embodiments, the duration of inflammation is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the duration of inflammation in the same subject prior to exposure to the composition or nutritional supplement.

In some embodiments, the composition and/or nutritional supplement reduces the frequency of one or more symptoms of inflammation in the subject. In some embodiments, the inflammation symptom frequency is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the inflammation symptom frequency in the same subject prior to exposure to the composition or nutritional supplement. In some embodiments, the inflammation symptom frequency is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the inflammation symptom frequency in the same subject prior to exposure to the composition or nutritional supplement.

In some embodiments, the composition and/or nutritional supplement reduces the duration of one or more symptoms of inflammation in the subject. In some embodiments, the inflammation symptom duration is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the inflammation symptom duration in the same subject prior to exposure to the composition or nutritional supplement. In some embodiments, the inflammation symptom duration is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the inflammation symptom duration in the same subject prior to exposure to the composition or nutritional supplement.

In some embodiments, the composition and/or nutritional supplement reduces the frequency of diarrhea in the subject. In some embodiments, the frequency of diarrhea is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the frequency of diarrhea in the subject prior to exposure to the composition or nutritional supplement. In some embodiments, the frequency of diarrhea is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the frequency of diarrhea in the same subject prior to exposure to the composition or nutritional supplement.

In some embodiments, the composition and/or nutritional supplement reduces the duration over which diarrhea occurs in the subject. In some embodiments, the duration over which diarrhea occurs is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the duration over which diarrhea occurs in the same subject prior to exposure to the composition or nutritional supplement. In some embodiments, the duration over which diarrhea occurs is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the duration over which diarrhea occurs in the same subject prior to exposure to the composition or nutritional supplement.

In some embodiments, the composition and/or nutritional supplement reduces the frequency of vomiting in the subject. In some embodiments, the frequency of vomiting is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the frequency of vomiting in the same subject prior to exposure to the composition or nutritional supplement. In some embodiments, the frequency of vomiting is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the frequency of vomiting in the same subject prior to exposure to the composition or nutritional supplement.

In some embodiments, the composition and/or nutritional supplement reduces the duration over which vomiting occurs in the subject. In some embodiments, the duration over which vomiting occurs is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the duration over which vomiting occurs in the same subject prior to exposure to the composition or nutritional supplement. In some embodiments, the duration over which vomiting occurs is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the duration over which vomiting occurs in the same subject prior to exposure to the composition or nutritional supplement.

In some embodiments, the composition and/or nutritional supplement reduces the frequency of itching, paw licking, and/or head shaking in the subject. In some embodiments, the frequency of itching, paw licking, and/or head shaking is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the frequency of itching, paw licking, and/or head shaking in the same subject prior to exposure to the composition or nutritional supplement. In some embodiments, the frequency of itching, paw licking, head shaking is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the frequency of itching, paw licking, and/or head shaking in the same subject prior to exposure to the composition or nutritional supplement.

In some embodiments, the composition and/or nutritional supplement reduces the duration over which itching, paw licking, and/or head shaking occurs in the subject. In some embodiments, the duration over which itching, paw licking, and/or head shaking occurs is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to the duration over which itching occurs in the same subject prior to exposure to the composition or nutritional supplement. In some embodiments, the duration over which itching, paw licking, and/or head shaking occurs is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to the duration over which itching, paw licking, and/or head shaking occurs prior to prior to exposure to the composition or nutritional supplement.

In some embodiments, the composition and/or nutritional supplement reduces the frequency of one or more symptoms of allergy in the subject. In some embodiments, the allergy symptom frequency is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 105-fold or more as compared to allergy symptom frequency in the same subject prior to exposure to the composition or nutritional supplement. In some embodiments, allergy symptom frequency is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to allergy symptom frequency in the same subject prior to exposure to the composition or nutritional supplement.

In some embodiments, the composition and/or nutritional supplement reduces the duration of one or more symptoms of allergy in the subject. In some embodiments, the allergy symptom duration is reduced by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more as compared to allergy symptom duration in the same subject prior to exposure to the composition or nutritional supplement. In some embodiments, allergy symptom duration is reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% or more as compared to allergy symptom duration in the same subject prior to exposure to the composition or nutritional supplement.

### Production of Indole-Containing Molecules

Bacterial strains of the compositions and nutritional supplements described herein were identified based on the ability to produce one or more indole-containing molecules during fermentation in the presence of L-tryptophan (e.g., fermentation in a conventional rich medium). Indole-containing molecules act as agonists of aryl hydrocarbon receptor (AhR) signaling pathways, which have been shown to be involved in maintaining a healthy intestinal immune system and combating intestinal inflammation in vivo.

Non-limiting examples of such indole-containing metabolites are shown in FIG. 1A, and include indoleacetic acid, indoleacrylic acid, indolepropionic acid, indolepyruvic acid, indoleacetamide, indole-3-acetic acid, indolealdehyde, indole, tryptamine, 3-indoleacrylic acid, 3-indolepropionic acid, indoxylsulfate, indole, 3-indolelactic acid, 3-indolepyruvic acid, 3-indoleacetamide, 3-indoleacetaldehyde, 3-indoleacetic acid, 3-indolealdehyde, 3-indoleethanol (also referred to as indole-3-ethanol (IE) or tryptophol), skatole, and N-acetyltryptamine. In some embodiments, fermentation by bacterial strains of a bacterial mixture in the presence of L-tryptophan produces 1, 2, 3, 4, 4, 5, 6, 7, 8, 9, 10, 11, 12, 13, 14, 15, 16, 17, 18, 19, or 20 indole-containing molecules with distinct structures. In some embodiments, fermentation by strains of a bacterial mixture produces 1-4, 4-8, 8-12, 12-16, or 16-20 indole-containing molecules with distinct structures. In some embodiments, fermentation by strains of a bacterial mixture produces a molecule selected from indole-3-acetic acid, indolepropionic acid, indole-3-aldehyde, and indoleacetic acid. In some embodiments, fermentation by strains of a bacterial mixture produces 2, 3, or 4 molecules selected from indole-3-acetic acid, indolepropionic acid, indole-3-aldehyde, and indoleacetic acid. In some embodiments, fermentation by strains of a bacterial mixture produces indole-3-acetic acid, indolepropionic acid, indole-3-aldehyde, and indoleacetic acid.

In some embodiments, fermentation produces indole-3-acetic acid in a concentration of 0.5-100, 0.5-80, 0.5-60, 0.5-40, 0.5-20, 0.5-15, 0.5-10, 2-90, 2-70, 2-50, 2-30, 2-20, 2-15, 2-10, 4-60, 4-40, 4-30, 4-20, 4-15, 4-10, 5-50, 5-40, 5-30, 5-20, 5-15, or 5-10. In some embodiments, fermentation produces a concentration of indole-3-acetic acid of 8.5 μM or more. In some embodiments, fermentation produces a concentration of indole-3-acetic acid of 8.5-100 M. In some embodiments, fermentation produces indole-3-acetic acid in a concentration of about 8.5 μM.

In some embodiments, fermentation produces indoleacetic acid in a concentration of 0.5-100, 0.5-80, 0.5-60, 0.5-40, 0.5-20, 0.5-15, 0.5-10, 2-90, 2-70, 2-50, 2-30, 2-20, 2-15, 2-10, 4-60, 4-40, 4-30, 4-20, 4-15, 4-10, 5-50, 5-40, 5-30, 5-20, 5-15, or 5-10 8.5 μM. In some embodiments, fermentation produces indoleacetic acid in a concentration of 10 μM or more. In some embodiments, fermentation produces indoleacetic acid in a concentration of 10-100 μM. In some embodiments, fermentation produces indoleacetic acid in a concentration of about 10 μM.

In some embodiments, fermentation produces indole-3-aldehyde in a concentration of 0.1-10, 0.1-9, 0.1-8, 0.1-7, 0.1-6, 0.1-5, 0.1-4, 0.1-3, 0.1-2, 0.1-1.5, 0.2-10, 0.2-9, 0.2-8, 0.2-7, 0.2-6, 0.2-5, 0.2-4, 0.2-3, 0.2-2, 0.2-1.5, 0.3-10, 0.3-9, 0.3-8, 0.3-7, 0.3-6, 0.3-5, 0.3-4, 0.3-3, 0.3-2, 0.3-1.5, 0.4-10, 0.4-9, 0.4-8, 0.4-7, 0.4-6, 0.4-5, 0.4-4, 0.4-3, 0.4-2, 0.4-1.5, 0.5-10, 0.5-9, 0.5-8, 0.5-7, 0.5-6, 0.5-5, 0.5-4, 0.5-3, 0.5-2, or 0.5-1.5 M. In some embodiments, fermentation produces indole-3-aldehyde in a concentration of 1.0 μM or more. In some embodiments, produces indole-3-aldehyde in a concentration of 1.0-20 μM. In some embodiments, fermentation produces indole-3-aldehyde in a concentration of 1.0 μM.

In some embodiments, fermentation produces indolepropionic acid in a concentration of 0.1-10, 0.1-9, 0.1-8, 0.1-7, 0.1-6, 0.1-5, 0.1-4, 0.1-3, 0.1-2, 0.1-1.5, 0.2-10, 0.2-9, 0.2-8, 0.2-7, 0.2-6, 0.2-5, 0.2-4, 0.2-3, 0.2-2, 0.2-1.5, 0.3-10, 0.3-9, 0.3-8, 0.3-7, 0.3-6, 0.3-5, 0.3-4, 0.3-3, 0.3-2, 0.3-1.5, 0.4-10, 0.4-9, 0.4-8, 0.4-7, 0.4-6, 0.4-5, 0.4-4, 0.4-3, 0.4-2, 0.4-1.5, 0.5-10, 0.5-9, 0.5-8, 0.5-7, 0.5-6, 0.5-5, 0.5-4, 0.5-3, 0.5-2, or 0.5-1.5 μM. In some embodiments, fermentation produces indolepropionic acid in a concentration of 1.0 μM or more. In some embodiments, fermentation produces indolepropionic acid in a concentration of 1.0-20 μM. In some embodiments, fermentation produces indolepropionic acid in a concentration of 1.0 μM.

Production of indole-containing molecules by bacterial strains generally occurs by modification of L-tryptophan, as shown in FIG. 1A. The abundance of L-tryptophan may thus affect the amounts of one or more indole-containing molecules produced by fermentation. Accordingly, one or more bacterial strains of a bacterial mixture, composition, or nutritional supplement described herein may produce L-tryptophan in the presence of protein. Production of free L-tryptophan in the presence of protein (e.g., by metabolism of proteins into free amino acids, including L-tryptophan) promotes the availability of L-tryptophan for metabolism into other indole-containing molecules, thereby preventing L-tryptophan concentration from becoming rate-limiting.

Not all bacterial strains of a bacterial mixture must produce L-tryptophan, as some bacterial strains may produce L-tryptophan while others metabolize L-tryptophan to produce indole-containing molecules. Accordingly, in some embodiments, one or more bacterial strains of a bacterial mixture (e.g., in a composition or nutritional supplement) described herein produces L-tryptophan in the presence of protein. In some embodiments, each of the bacterial strains produces L-tryptophan in the presence of protein. In some embodiments, 2, 3, 4, 5, 6, 7, 8, 9, 10, 11, 12, 13, 14, 15, 16, 17, 18, 19, or 20 bacterial strains produce L-tryptophan in the presence of protein. Determining whether a bacterial strain produces L-tryptophan in the presence of protein may be conducted by any method known in the art, such as detecting L-tryptophan in a medium during or after bacterial fermentation.

The bacterial strains of the bacterial mixture are preferably in a live (e.g., viable) state or in a mixture of live and inactivated (e.g., killed, not viable) states. As will be appreciated by one of ordinary skill in the art, bacterial cells may be considered live or living if the cells are metabolically active, e.g., have a detectable level of metabolic activity. Being metabolically active does not require proliferation or replication of the cells. Methods of evaluating whether a bacterial strain is living and is metabolically active are known in the art, for example, viability assays, detection of ATP measurements, membrane potential, respiratory activity, uptake of dyes. See, e.g., Emerson et al. Microbiome (2017) 5:86. In some embodiments, one or more of the bacterial strains of the bacterial mixture are in a live state and maintain viability in the composition or nutritional supplement, for example following production and/or storage conditions.

In some embodiments, at least one (e.g., 1, 2, 3, 4, 5, 6, 7, 8, 9, or 10) of the bacterial strains in the composition is a spore-former. In some embodiments, at least one (e.g., 1, 2, 3, 4, 5, 6, 7, 8, 9, or 10) of the bacterial strains in the composition is in spore form. In some embodiments, at least one (e.g., 1, 2, 3, 4, 5, 6, 7, 8, 9, or 10) of the bacterial strains in the composition is a non-spore former. In some embodiments, at least one (e.g., 1, 2, 3, 4, 5, 6, 7, 8, 9, or 10) of the bacterial strains in the composition is in vegetative form. As discussed above, spore forming bacteria can also be in vegetative form. In some embodiments, at least one (e.g., 1, 2, 3, 4, 5, 6, 7, 8, 9, or 10) of the bacterial strains in the composition is in spore form and at least one (e.g., 1, 2, 3, 4, 5, 6, 7, 8, 9, or 10) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form. In some embodiments, each of the bacterial strains is in vegetative form.

It is envisioned that the bacterial strains of the compositions described herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

In some embodiments, the bacterial strains of the bacterial mixture provided herein are in vegetative form, meaning the bacterial cells are not actively growing and/or reproducing. In some embodiments, at least one (e.g., 1, 2, 3, 4, 5, or more) of the bacterial strains of the bacterial mixture is in vegetative form. In some embodiments, each of the bacterial strains of the bacterial mixture is in vegetative form. It will be appreciated that live bacterial strains may be viable but may not be actively growing/replicating. For example, following utilization of the sugar source in a fermentation medium, bacterial strains may slow or halt active replication due to reduced levels of available nutrients.

In some embodiments, bacterial strains of the bacterial communities provided herein are living and are alive in the nutritional supplement. Viability can be determined by quantifying the colony forming units (CFU), for example by plating a sample of the bacterial community, or composition or nutritional supplement, on a nutritive agar medium. The number of colony forming units corresponds to the number of viable bacterial cells in the sample tested such as the community or composition or nutritional supplement.

The bacterial strains described herein may be obtained from or derived from any source known in the art, such as from a food source or an environmental source. As used herein, the term “derived from” in the context of bacterial strains derived from a particular source refers to obtaining a bacterial strain from the source, which may involve isolating and/or propagating cells of a bacterial strain. In some embodiments, the bacterial strains are further manipulated, such as purified and/or analyzed, prior to use in the composition or nutritional supplements and methods described herein. As will be evident to one of ordinary skill in the art, reference to a bacterial strain or cells of a bacterial strain that is derived from a particular source encompasses progeny cells thereof.

The bacterial strains of the bacterial community may be derived from a fermented food or beverage, such as cultured milk and yogurt, natto, cheese, kombucha, wine, beer, cider, miso, kimchi, sauerkraut, fermented sausage, among others. Additionally, bacterial strains may be derived from cultured plants or plant protein isolates, including plants or proteins isolated from cercal grains (e.g., oats), oil seeds (e.g., sunflower seeds), legumes, pulses, beans, broad beans, faba beans, peas, chickpeas, cow peas, pigeon peas, lentils, Bambara beans, vetches, and lupins. In some embodiments, one or more bacterial strains are derived from a fermented meat. In some embodiments, one or more bacterial strains are derived from salami. In some embodiments, one or more bacterial strain are derived from a fermented vegetable mix. In some embodiments, one or more bacterial strains are derived from kimchi.

In some embodiments, at least one bacterial strain of the bacterial mixture may be. In some embodiments, at least one bacterial strain of the bacterial mixture may be isolated. Any of the bacterial strains described herein may be isolated and/or purified, for example, from a source such as a food source (e.g., a fermented food or beverage product) or an environmental source.

In any of the compositions described herein, including nutritional supplements comprising bacterial strains, the bacterial strains may be in any form, for example in an aqueous form, such as a solution or a suspension, embedded in a semi-solid form, in a powdered form, or freeze-dried form. In some embodiments, the bacterial strains are lyophilized or spray-dried. In some embodiments, at least one bacterial strain of the bacterial mixture is in a lyophilized form. In some embodiments, each of bacterial strains of the bacterial mixture is in a lyophilized form. In some embodiments, the composition or the bacterial strains are lyophilized. In some embodiments, a subset of the bacterial strains is lyophilized. In some embodiments, each of the bacterial strains are lyophilized. Methods of lyophilizing compositions, specifically compositions comprising bacteria, are known in the art. See, e.g., U.S. Pat. Nos. 3,261,761; 4,205,132; and PCT Publication Nos. WO 2014/029578 and WO 2012/098358, herein incorporated by reference in their entirety. The bacteria may be lyophilized as a combination and/or the bacteria may be lyophilized separately and combined prior to incorporation into a composition or nutritional supplement. A bacterial strain may be combined with a nutrient or carrier prior to combining it with the other bacterial strain or multiple lyophilized bacteria may be combined while in lyophilized form and the mixture of bacteria, once combined may subsequently be combined with a nutrient and/or excipient. In some embodiments, the bacterial strain is a lyophilized cake. In some embodiments, the compositions comprising the one or more bacterial strains are a lyophilized cake. In such embodiments, the bacterial strain(s) may be rehydrated or suspended and/or cultured prior to use in the methods described herein. In some embodiments, the bacterial strain(s) in lyophilized form are used directly, for example, without rehydrating or suspension (e.g., directly added to a medium).

In some embodiments, one of or more of the bacterial strains of the compositions has been spray-dried. The process of spray-drying refers to production of a dry powder from a liquid comprising bacterial compositions. (See, e.g., Ledet et al., Spray-Drying of Pharmaceuticals in “Lyophilized Biologics and Vaccines” pages 273-194, Springer). In general, the process involves rapidly drying the bacterial compositions with a hot gas. A bacterial strain may be combined with an excipient or nutrient prior to combining it with the other bacterial strains or multiple spray-dried bacterial strains may be combined while in spray-dried form and the mixture of bacterial strains, once combined, may be subsequently combined with an excipient, carrier, or nutrient.

In some embodiments, the compositions comprising bacterial strains are formulated for oral delivery. In some embodiments, the compositions comprising bacterial strains are formulated for delivery to the intestines (e.g., the small intestine and/or the colon). In some embodiments, the compositions comprising bacterial strains are formulated for delivery to the intestines (e.g., the small intestine and/or the colon) such that the bacterial strains maintain viability and are able to colonize (engraft) the intestines of the subject.

In some embodiments, the composition comprising bacterial strains may be formulated with an enteric coating that increases the survival of the bacteria through the harsh environment in the stomach. Such an enteric coating may comprise one or more pH-sensitive enteric polymers. The enteric coating is one which resists the action of gastric juices in the stomach so that the bacteria of the composition therein will pass through the stomach and into the intestines. The enteric coating may readily dissolve when in contact with intestinal fluids, so that the bacteria enclosed in the coating will be released in the intestinal tract. Enteric coatings may consist of polymers and copolymers well known in the art, such as commercially available EUDRAGIT (Evonik Industries). Sec e.g., Zhang, AAPS PharmSciTech (2016) 17 (1), 56-67.

The bacterial strains can be manufactured using fermentation techniques well known in the art. In some embodiments, the bacteria are propagated or manufactured using liquid fermenters, which can support the rapid growth of bacterial species. The fermenters may be, for example, stirred tank reactors or disposable wave bioreactors. Culture media such as BL media and EG media, or similar versions of these media, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the bacterial strains can be manufactured using fermentation techniques and conditions to promote the formation of bacterial spores or enhance the formation of bacterial spores. Methods of enhancing spore formation and yield are known in the art and include, for example, medium compositions, such as particular carbon sources (e.g., rice straw hydrolase), sodium chloride, peptone, yeast extract, ammonium phosphate; oxygen supply; growth temperature; and drying methods (e.g., hot air drying, vacuum freeze drying). See, e.g., Yin et al. Lett. Appl. Microbiol. (2021) 72(2): 149-156; Penna et al. PDA J. Pharm. Sci. Technol. (1998) 52(5): 198-208; Boniolo et al. Appl. Micrbiol. Biotechnol. (2012) 94(3): 625-36; Li et al. Front. Nutr. (2022) 9:1025248; Li et. Al. J. Ind. Microbiol. Biotechnol. (2022) 49(4); Petrillo et al. Microb. Cell Fact. (2020) 19(1): 185; and Tavares et al. Curr. Microbiol. (2013) 66(3): 279-85; each of which is incorporated by reference herein.

As used herein, the term “isolated” refers to a bacterial cell or bacterial strain that has been separated from one or more undesired components, such as other bacterial cells or bacterial strains, one or more components of a growth medium, one or more components of a food or beverage product. In some embodiments, the bacterial strains are substantially isolated from a source such that other components of the source are not detected. In some embodiments, a bacterial strain is isolated or purified from a sample and then cultured (grown, propagated) under the appropriate conditions for replication. The bacterial strain that is grown under appropriate conditions for replication can subsequently be isolated/purified from the culture in which it is grown.

Also within the scope of the present disclosure are isolated bacterial communities. In this context, the term “isolated” refers to a bacterial community that has been separated from one or more undesired component, such as other bacterial cells, bacterial strains, bacterial communities, one or more component of a growth medium, one or more component of a food or beverage product, and/or one or more component of a sample, such as an environmental sample. In some embodiments, the bacterial communities are substantially isolated from a source such that other components of the source are not detected. In some embodiments, bacterial strains are individually cultured and then combined forming the bacterial community. In some embodiments, the bacterial strains are combined forming a bacterial community, which is then cultured (grown, propagated) collectively, as a community under the appropriate conditions for replication. The bacterial strain that is grown under appropriate conditions for replication can subsequently be isolated/purified from the culture in which it is grown.

The specific bacterial strains selected and combined to form the bacterial communities described herein may have beneficial properties when present or used in combination, as compared to bacterial communities that contain different combinations of bacterial strains or to bacterial strains alone (not in combination). For example, the bacterial communities described herein, when compared to single strains or other bacterial communities, produce an indole-containing molecule in a concentration that is at least 1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, or up to 50-fold higher than a bacterial community containing a different combination of bacterial strains or the bacterial strains when present or used alone under similar fermentation conditions. In some embodiments, the bacterial community produces an indole-containing molecule at a concentration that is about 1.5-5-fold higher than a bacterial community containing a different combination of bacterial strains or the bacterial strains when present or used alone under similar fermentation conditions.

In some embodiments, the bacterial communities described herein, when compared to single strains or other bacterial communities, may replicate more quickly and reach a higher biomass during fermentation. In some embodiments, the bacterial communities described herein replicate at a rate that is at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, or up to 50-fold higher than a bacterial community containing a different combination of bacterial strains or the bacterial strains when present or used alone under similar fermentation conditions. In some embodiments, the bacterial communities described herein replicate at a rate that is about 2-3-fold higher than a bacterial community containing a different combination of bacterial strains or the bacterial strains when present or used alone under similar fermentation conditions.

In some embodiments, the bacterial communities described herein reach a biomass that is at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 300-fold, 400-fold or up to 500-fold higher than the biomass that can be reached with a bacterial community containing a different combination of bacterial strains or with a bacterial strain alone under similar fermentation conditions. In some embodiments, the bacterial communities described herein reach a biomass that is about 5-10-fold higher than the biomass that can be reached with a bacterial community containing a different combination of bacterial strains or with a bacterial strain alone under similar fermentation conditions.

Methods of assessing the replication rate of a bacterial strain or community thereof, as well as methods of quantifying biomass, are known in the art. See, e.g., Brown et al., Nature Biotechnology (2016) 34: 1256-1263.

As another example, the bacterial communities described herein may ferment more quickly than other bacterial communities or than single bacterial strains used alone. The rate of fermentation of a bacterial community and/or individual strain can be assessed by any means known in the art, such as the utilization of a sugar source, level of a sugar in the fermentation medium, change in pH of the medium, generation of a fermentation product (e.g., organic acids, CO₂). In some embodiments, the bacterial communities described herein ferment at a rate that is at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, or up to 50-fold higher than a bacterial community containing a different combination of bacterial strains or single bacterial strains when used alone under similar fermentation conditions. In some embodiments, the bacterial communities described herein ferment at a rate that is about 2-3-fold higher than a bacterial community containing a different combination of bacterial strains or single bacterial strains when used alone under similar fermentation conditions.

In some embodiments, the bacterial strains used in any of the bacterial mixtures described herein are preferably “generally recognized as safe” (GRAS) or approved as food additives according to the U.S. Food and Drug Administration. Sec, e.g., Federal Food, Drug, and Cosmetic Act, sections 201 (s) and 409.

### Stability of Bacterial Strains

The compositions and nutritional supplements as described herein may be shelf-stable. “Shelf-stable” refers to stability and product integrity when a product is maintained at ambient temperatures (about 20°−22° C.) for an extended period of time. In some embodiments, a composition or nutritional supplement is stable at ambient temperature (i.e., between about 20°−22° C.) for at least or about 1 week, at least or about 2 weeks, at least or about 3 weeks, at least or about 4 weeks, at least or about 5 weeks, at least or about 6 weeks, at least or about 7 weeks, at least or about 8 weeks, at least or about one month, at least or about two months, at least or about three months, at least or about four months, at least or about five months, at least or about six months, at least or about seven months, at least or about eight months, at least or about nine months, at least or about ten months, at least or about eleven months, at least or about twelve months, at least or about one year, at least or about two years, at least or about three years, at least or about four years, at least or about five years, or longer.

In some embodiments, the composition or nutritional supplement has a water activity level (a_{w}) of 0.25 or more. In some embodiments, the a_{w} is 0.9 or less. In some embodiments, the a_{w} is 0.8 or less. Water activity refers to the amount of free water in a composition, defined as the ratio between water vapor pressure in a material (p) and the vapor pressure of pure water (po) at the same temperature. Sec, e.g., Esbelin et al., Food Microbiol. 2018. 69:82-88. The water content of a composition affects multiple characteristics, including the stability of bacterial strains, and nucleic acids, proteins, and other components thereof, which may be subject to alteration (e.g., degradation) by hydrolysis. Compositions with a low water content, however, are dry and have an undesirable texture, which reduces the sensory desirability of a composition nutritional supplement. In some embodiments, the compositions or nutritional supplements have an a_{w} of 0.25 or more, 0.3 or more, 0.4 or more, 0.5 or more, 0.6 or more, 0.7 or more, 0.8 or more, or 0.9 or more. In some embodiments, the a_{w} is 0.3 or more. In some embodiments, the a_{w} is 0.4 or more. In some embodiments, the a_{w} is 0.5 or more. In some embodiments, the a_{w} is 0.6 or more. In some embodiments, the a_{w} is 0.7 or more. In some embodiments, the compositions or nutritional supplements have an a″ of 1.0 or less, 0.9 or less, 0.8 or less, 0.7 or less, 0.6 or less, 0.5 or less, 0.4 or less, or 0.3 or less. In some embodiments, the a_{w} is 0.8 or less. In some embodiments, the a_{w} is 0.7 or less. In some embodiments, the a_{w} is 0.6 or less. In some embodiments, the a_{w} is 0.5 or less. In some embodiments, the a_{w} is 0.4 or less. In some embodiments, the a_{w} is 0.3 or less. In some embodiments, the a_{w} is 0.3-0.8, 0.3-0.7, 0.3-0.6, 0.3-0.5, 0.3-0.4, 0.4-0.8, 0.4-0.7, 0.4-0.6, 0.4-0.5, 0.5-0.8, 0.5-0.7, 0.5-0.6, 0.6-0.8, 0.6-0.7, or 0.7-0.8. In some embodiments, the a_{w} is 0.25-0.9, 0.25-0.8, 0.25-0.7, 0.25-0.6, 0.25-0.5, 0.25-0.4, 0.4-0.7, 0.4-0.6, or 0.4-0.5.

In some embodiments, compositions or nutritional supplements allow for recovery of at least 1%, at least 10%, at least 20%, at least 30%, at least 40%, at least 50%, at least 60%, at least 70%, at least 80%, at least 90%, up to 100% of the colony forming units of the composition or nutritional supplement over a period of time. In some embodiments, the period of time is at least 1 week, at least 2 weeks, at least 3 weeks, at least 4 weeks, at least 6 weeks, at least 8 weeks, at least 2 months, at least 3 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In some embodiments, the period of time is at least 1 week. In some embodiments, the period of time is at least 2 weeks. In some embodiments, the period of time is at least 3 weeks. In some embodiments, the period of time is at least 4 weeks. In some embodiments, the period of time is at least 5 weeks. In some embodiments, the period of time is at least 6 weeks. In some embodiments, the period of time is at least 7 weeks. In some embodiments, the period of time is at least 8 weeks. In some embodiments, the period of time is at least 2 months. In some embodiments, the period of time is at least 3 months. In some embodiments, the period of time is at least 4 months. In some embodiments, the period of time is at least 5 months. In some embodiments, the period of time is at least 6 months. In some embodiments, the period of time is at least 7 months. In some embodiments, the period of time is at least 8 months. In some embodiments, the period of time is at least 9 months. In some embodiments, the period of time is at least 10 months. In some embodiments, the period of time is at least 11 months. In some embodiments, the period of time is at least 12 months. In some embodiments, the period of time is at least 13 months. In some embodiments, the period of time is at least 14 months. In some embodiments, the period of time is at least 15 months. In some embodiments, the period of time is at least 16 months. In some embodiments, the period of time is at least 17 months. In some embodiments, the period of time is at least 18 months. In some embodiments, the period of time is at least 19 months. In some embodiments, the period of time is at least 20 months. In some embodiments, the period of time is at least 21 months. In some embodiments, the period of time is at least 22 months. In some embodiments, the period of time is at least 23 months. In some embodiments, the period of time is at least 24 months. In some embodiments, the period of time is 1-24, 1-18, 1-12, 1-9, 1-6, 6-24, 6-18, 6-12, 12-24, 12-18, or 18-24 months.

In some embodiments, the percentage of recovered colony forming units is determined by comparing a number of colony forming units of bacteria (e.g., of a bacterial strain or total bacteria) at a first time point relative to the number of colony forming units of bacteria (e.g., of a bacterial strain or total bacteria) at a second time point over a period of time. For example, a 50% recovery or preservation of 50% of bacteria indicates that half of the bacteria remained viable over the period of time; and a 100% recovery or preservation of 100% of bacteria indicates that all (or substantially all) bacteria remained viable over the period of time.

In some embodiments, the compositions or nutritional supplements described herein may also have increased stability or product integrity at an elevated temperature, e.g., above about 23° C. In some embodiments, the compositions or nutritional supplements described herein have increased stability or product integrity at a temperature higher than or about 23° C., higher than or about 24° C., higher than or about 25° C., higher than or about 26° C., higher than or about 27° C., higher than or about 28° C., about 29° C., higher than or about 30° C., higher than or about 31° C., higher than or about 32° C., higher than or about 33° C., higher than or about 34° C., higher than or about 35° C., higher than or about 36° C., higher than or about 37° C., higher than or about 38° C., higher than or about 39° C., higher than or about 40° C. or higher.

In some embodiments, the composition or nutritional supplement is stable at an elevated temperature, e.g., above or about 23° C., for at least or about 1 week, at least or about 2 weeks, at least or about 3 weeks, at least or about 4 weeks, at least or about 5 weeks, at least or about 6 weeks, at least or about 7 weeks, at least or about 8 weeks, at least or about one month, at least or about two months, at least or about three months, at least or about four months, at least or about five months, at least or about six months, at least or about seven months, at least or about eight months, at least or about nine months, at least or about ten months, at least or about eleven months, at least or about twelve months, at least or about one year, at least or about two years, at least or about three years, at least or about four years, at least or about five years, or longer.

In some embodiments, the composition or nutritional supplement is stable at lower than ambient temperature, e.g., below 20° C., for at least or about 1 week, at least or about 2 weeks, at least or about 3 weeks, at least or about 4 weeks, at least or about 5 weeks, at least or about 6 weeks, at least or about 7 weeks, at least or about 8 weeks, at least or about one month, at least or about two months, at least or about three months, at least or about four months, at least or about five months, at least or about six months, at least or about seven months, at least or about eight months, at least or about nine months, at least or about ten months, at least or about eleven months, at least or about twelve months, at least or about one year, at least or about two years, at least or about three years, at least or about four years, at least or about five years, or longer. In some embodiments, the composition or nutritional supplement is stable during storage at 2° C. to 8° C. In some embodiments, the composition or nutritional supplement is stable during storage at 8° C. to 20° C. In some embodiments, the composition or nutritional supplement is stable during storage at 20° C. to 40° C. In some embodiments, the composition or nutritional supplement is stable during storage at 25° C.

Determining whether a composition or nutritional supplement is stable or the product integrity is intact will be evident to one of ordinary skill in the art and may involve using methods known in the art. For example, in some embodiments, the stability of the composition or nutritional supplement under particular conditions involves evaluating, for example, flavor profile, color profile, amounts of CFUs of bacterial strains, pH, and/or detection of contaminants. In general, a composition or nutritional supplement is considered to be “stable” if one or more properties (e.g., CFU of the bacterial strains, flavor profile, pH, etc) of the composition or nutritional supplement does not substantially change under particular conditions.

In some embodiments, the composition or nutritional supplements comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacterial strains per gram of the composition or nutritional supplement. In some embodiments, the composition or nutritional supplements comprise at least or about 10, at least or about 10², at least or about 10³, at least or about 10⁴, at least or about 10⁵, at least or about 10⁶, at least or about 10⁷, at least or about 10⁸, at least or about 10⁹, at least or about 10¹⁰, at least or about 10¹¹, at least or about 10¹², at least or about 10¹³ or more of each of the bacterial strains per gram of the composition or nutritional supplement.

In some embodiments, the composition or nutritional supplement contains between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10 and about 10¹², between about 10² and about 10¹², between about 10³ and about 10¹², between about 10⁴ and about 10¹², between about 10⁵ and about 10¹², between about 10⁶ and about 10¹², between about 10⁷ and about 1012, between about 10⁸ and about 10¹², between about 10⁹ and about 10¹², between about 10¹⁰ and about 10¹², between about 10¹¹ and about 10¹², between about 10 and about 10¹¹, between about 10² and about 10¹¹, between about 10³ and about 10¹³, between about 10⁴ and about 10¹³, between about 10⁵ and about 10¹³, between about 10⁶ and about 10¹³, between about 10⁷ and about 10¹¹, between about 10⁸ and about 10¹¹, between about 10⁹ and about 1011, between about 10¹⁰ and about 10¹¹, between about 10 and about 10¹⁰, between about 10² and about 10¹⁰, between about 10³ and about 10¹⁰, between about 10⁴ and about 10¹⁰, between about 10⁵ and about 10¹⁰, between about 10⁶ and about 10¹⁰, between about 10⁷ and about 10¹⁰, between about 10⁸ and about 10¹⁰, between about 10⁹ and about 10¹⁰, between about 10 and about 10⁹, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10 and about 10⁸, between about 10² and about 10⁸, between about 10³ and about 10⁸, between about 10⁴ and about 10⁸, between about 10⁵ and about 10⁸, between about 10⁶ and about 10⁸, between about 10⁷ and about 10⁸, between about 10 and about 10⁷, between about 10² and about 10⁷, between about 10³ and about 10⁷, between about 10⁴ and about 10⁷, between about 10⁵ and about 10⁷, between about 10⁶ and about 10⁷, between about 10 and about 10⁶, between about 10² and about 10⁶, between about 10³ and about 10⁶, between about 10⁴ and about 10⁶, between about 10⁵ and about 10⁶, between about 10 and about 10⁵, between about 10² and about 10⁵, between about 10³ and about 10⁵, between about 10⁴ and about 10⁵, between about 10 and about 10⁴, between about 10² and about 10⁴, between about 10³ and about 10⁴, between about 10 and about 10³, between about 10² and about 10³, or between about 10 and about 10² of each of the bacterial strains per gram of composition or nutritional supplement. In some embodiments, the composition or nutritional supplement contains between 10⁵ and 10⁶ of each of the bacterial strains per gram of composition or nutritional supplement. Each of the bacterial strains of the bacterial mixture may be present in the same amount or in different amounts.

In some embodiments, the composition or nutritional supplements comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacterial cells (CFU) of all bacterial strains per gram composition or nutritional supplement. In some embodiments, the composition or nutritional supplements comprise at least 10, at least 10², at least 10³, at least 10⁴, at least 10⁵, at least 10⁶, at least 10⁷, at least 10⁸, at least 109, at least 10¹⁰, at least 1011, at least 1012, at least 10¹³ or more of total bacterial cells (CFU) of all bacterial strains per gram composition or nutritional supplement.

In some embodiments, the composition or nutritional supplement contains between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10 and about 10¹², between about 10² and about 10¹², between about 10³ and about 10¹², between about 10⁴ and about 10¹², between about 10⁵ and about 10¹², between about 10⁶ and about 10¹², between about 10⁷ and about 1012, between about 10⁸ and about 10¹², between about 10⁹ and about 10¹², between about 10¹⁰ and about 10¹², between about 10¹¹ and about 10¹², between about 10 and about 10¹¹, between about 10² and about 10¹¹, between about 10³ and about 10¹³, between about 10⁴ and about 10¹³, between about 10⁵ and about 10¹³, between about 10⁶ and about 10¹³, between about 10⁷ and about 10¹¹, between about 10⁸ and about 10¹¹, between about 10⁹ and about 1011, between about 10¹⁰ and about 10¹¹, between about 10 and about 10¹⁰, between about 10² and about 10¹⁰, between about 10³ and about 10¹⁰, between about 10⁴ and about 10¹⁰, between about 10⁵ and about 10¹⁰, between about 10⁶ and about 10¹⁰, between about 10⁷ and about 10¹⁰, between about 10⁸ and about 10¹⁰, between about 10⁹ and about 10¹⁰, between about 10 and about 10⁹, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10 and about 10⁸, between about 10² and about 10⁸, between about 10³ and about 10⁸, between about 10⁴ and about 10⁸, between about 10⁵ and about 10⁸, between about 10⁶ and about 10⁸, between about 10⁷ and about 10⁸, between about 10 and about 10⁷, between about 10² and about 10⁷, between about 10³ and about 10⁷, between about 10⁴ and about 10⁷, between about 10⁵ and about 10⁷, between about 10⁶ and about 10⁷, between about 10 and about 10⁶, between about 10² and about 10⁶, between about 10³ and about 10⁶, between about 10⁴ and about 10⁶, between about 10⁵ and about 10⁶, between about 10 and about 10⁵, between about 10² and about 10⁵, between about 10³ and about 10⁵, between about 10⁴ and about 10⁵, between about 10 and about 10⁴, between about 10² and about 10⁴, between about 10³ and about 10⁴, between about 10 and about 10³, between about 10² and about 10³, or between about 10 and about 10² total bacterial cells (e.g., CFU) per gram of composition or nutritional supplement. In some embodiments, the composition or nutritional supplement contains between 10⁵ and 10⁶ total bacterial cells (e.g., CFU) per gram of composition or nutritional supplement. As discussed above, a bacterial strain may be present in the same amount or a different amount as compared to another bacterial strain.

### ENUMERATED EMBODIMENTS

1. A composition comprising a purified bacterial mixture comprising:
   (i) a first bacterial strain comprising a 16S rDNA sequence having at least 97% sequence identity to the nucleic acid sequence of SEQ ID NO: 1;
   (ii) a second bacterial strain comprising a 16S rDNA sequence having at least 97% sequence identity to the nucleic acid sequence of SEQ ID NO: 2; and
   (iii) a third bacterial strain comprising a 16S rDNA sequence having at least 97% sequence identity to the nucleic acid sequence of SEQ ID NO: 3, wherein the first bacterial strain and third bacterial strain are different bacterial strains.
2. The composition of embodiment 1, wherein the purified bacterial mixture consists of:
   (i) a first bacterial strain comprising a 16S rDNA sequence having at least 97% sequence identity to the nucleic acid sequence of SEQ ID NO: 1;
   (ii) a second bacterial strain comprising a 16S rDNA sequence having at least 97% sequence identity to the nucleic acid sequence of SEQ ID NO: 2; and
   (iii) a third bacterial strain comprising a 16S rDNA sequence having at least 97% sequence identity to the nucleic acid sequence of SEQ ID NO: 3.
3. The composition of embodiment 1 or 2, wherein
   (i) the first bacterial strain comprises a 16S rDNA sequence having the nucleic acid sequence of SEQ ID NO: 1;
   (ii) the second bacterial strain comprises a 16S rDNA sequence having the nucleic acid sequence of SEQ ID NO: 2; and
   (iii) the third bacterial strain comprises a 16S rDNA sequence having the nucleic acid sequence of SEQ ID NO: 3.
4. A composition comprising a purified bacterial mixture comprising:
   (i) a first bacterial strain comprising a 16S rDNA sequence having at least 97% sequence identity to the nucleic acid sequence of SEQ ID NO: 4;
   (ii) a second bacterial strain comprising a 16S rDNA sequence having at least 97% sequence identity to the nucleic acid sequence of SEQ ID NO: 5; and
   (iii) a third bacterial strain comprising a 16S rDNA sequence having at least 97% sequence identity to the nucleic acid sequence of SEQ ID NO: 6, wherein the first bacterial strain and third bacterial strain are different bacterial strains.
5. The composition of embodiment 4, wherein the purified bacterial mixture consists of:
   (i) a first bacterial strain comprising a 16S rDNA sequence having at least 97% sequence identity to the nucleic acid sequence of SEQ ID NO: 4;
   (ii) a second bacterial strain comprising a 16S rDNA sequence having at least 97% sequence identity to the nucleic acid sequence of SEQ ID NO: 5; and
   (iii) a third bacterial strain comprising a 16S rDNA sequence having at least 97% sequence identity to the nucleic acid sequence of SEQ ID NO: 6.
6 The composition of any one of the preceding embodiments, wherein
   (i) the first bacterial strain comprises a 16S rDNA sequence having the nucleic acid sequence of SEQ ID NO: 4;
   (ii) the second bacterial strain comprises a 16S rDNA sequence having the nucleic acid sequence of SEQ ID NO: 5; and
   (iii) the third bacterial strain comprises a 16S rDNA sequence having the nucleic acid sequence of SEQ ID NO: 6.
7. A composition comprising a purified bacterial mixture comprising a first bacterial strain belonging to the species Bacillus subtilis, a second bacterial strain belonging to the species Bacillus velezensis, and a third bacterial strain belonging to the species Bacillus subtilis, wherein the first bacterial strain and third bacterial strain are different bacterial strains.
8. The composition of embodiment 7, wherein the purified bacterial mixture consists of a first bacterial strain belonging to the species Bacillus subtilis, a second bacterial strain belonging to the species Bacillus velezensis, and a third bacterial strain belonging to the species Bacillus subtilis.
9. The composition of any one of the preceding embodiments, further comprising one or more additional bacterial strains.
10. The composition of any one of the preceding embodiments, wherein one or more of the additional bacterial strains belong to the genus Bacillus.
11. The composition of any one of the preceding embodiments, wherein one or more of the additional bacterial strains belong to the species Bacillus subtilis, Bacillus velezensis, Bacillus amyloliquefaciens, Bacillus lentus, Bacillus licheniformis, or Bacillus pumilus.
12. The composition of any one of the preceding embodiments, wherein the one or more of the additional bacterial strains comprise a 16S rDNA sequence with at least 97% sequence identity to the nucleic acid sequence of any one of SEQ ID NOs: 1-3.
13. The composition of any one of the preceding embodiments, wherein one or more of the additional bacterial strains comprise a 16S rDNA sequence with at least 97% sequence identity to the nucleic acid sequence of any one of SEQ ID NOs; 4-6.
14. The composition of any one of the preceding embodiments, wherein the composition produces one or more indole-containing molecules in the presence of L-tryptophan.
15. The composition of any one of the preceding embodiments, wherein the composition produces at least 4 indole-containing molecules in the presence of L-tryptophan.
16. The composition of any one of the preceding embodiments, wherein the indole-containing molecules are indoleacetic acid, indoleacrylic acid, indolepropionic acid, indolepyruvic acid, indoleacetamide, indole-3-acetic acid, indole-3-ethanol, indolealdehyde, indole, and/or tryptamine.
17. The composition of any one of the preceding embodiments, wherein the indole-containing molecules comprise indole-3-lactic acid, indole-3-aldehyde, indolepropionic acid, and/or indole-3-acetic acid.
18. The composition of any one of the preceding embodiments, wherein the composition produces indole-3-acetic acid in a concentration of 8.5 UM or more.
19. The composition of any one of the preceding embodiments, wherein the composition produces indoleacetic acid in a concentration of 10 μM or more.
20. The composition of any one of the preceding embodiments, wherein the composition produces indolepropionic acid in a concentration of 1.0 μM or more.
21. The composition of any one of the preceding embodiments, wherein the composition produces indole-3-aldehyde in a concentration of 1.0 μM or more.
22. The composition of any one of the preceding embodiments, wherein the composition produces L-tryptophan in the presence of protein.
23. The composition of any one of the preceding embodiments, wherein the composition reduces inflammation in a subject.
24. The composition of any one of the preceding embodiments, wherein the composition treats or prevents a symptom associated with inflammation in a subject.
25. The composition of any one of the preceding embodiments, wherein the composition treats or prevents diarrhea in a subject.
26. The composition of any one of the preceding embodiments, wherein the composition treats or prevents vomiting in a subject.
27. The composition of any one of the preceding embodiments, wherein the composition treats or prevents itching, paw licking, and/or head shaking in a subject.
28. The composition of any one of the preceding embodiments, wherein the composition treats or prevents allergy in a subject.
29. The composition of any one of the preceding embodiments, wherein the subject is a carnivore.
30. The composition of any one of the preceding embodiments, wherein the subject is a mammal.
31. The composition of any one of the preceding embodiments, wherein the subject is a domesticated animal.
32. The composition of any one of the preceding embodiments, wherein the subject is a rodent.
33. The composition of any one of the preceding embodiments, wherein the subject is a mouse, rat, guinea pig, chinchilla, or hamster.
34. The composition of any one of the preceding embodiments, wherein the subject is a dog, cat, rabbit, guinea pig, hamster, or ferret.
35. The composition of any one of the preceding embodiments, wherein the subject is a dog.
36. The composition of any one of the preceding embodiments, wherein the subject is a bovine, swine, llama, alpaca, sheep, or goat.
37. The composition of any one of the preceding embodiments, wherein the subject is a bird.
38. The composition of any one of the preceding embodiments, wherein the bacterial strains are lyophilized.
39. The composition of any one of the preceding embodiments, wherein the bacterial strains are spray-dried.
40. The composition of any one of the preceding embodiments, wherein the bacterial strains are in a dry, powdered form.
41. The composition of any one of the preceding embodiments, wherein one or more of the bacterial strains is in spore form.
42. The composition of any one of the preceding embodiments, wherein each of the bacterial strains is in spore form.
43. The composition of any one of any one of the preceding embodiments, wherein one or more of the bacterial strains is in vegetative form.
44. The composition of any one of the preceding embodiments, wherein each of the bacterial strains is in vegetative form.
45. The composition of any one of the preceding embodiments, wherein the composition has a water activity level (a_{w}) of 0.8 or less.
46. The composition of any one of the preceding embodiments, wherein at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the composition are viable after storage for a period of time.
47. The composition of any one of the preceding embodiments, wherein the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.
48. The composition of any one of the preceding embodiments, wherein the storage is conducted at 2° C. to 8° C., 8° C. to 20° C., or 20° C. to 40° C. 49. The composition of any one of the preceding embodiments, wherein the storage is conducted at a temperature of 25° C. or higher.
50. The composition of any one of the preceding embodiments, wherein the composition has a water activity level (a_{w}) of 0.25 or more, 0.3 or more, 0.4 or more, 0.5 or more, 0.6 or more, or 0.7 or more during the storage.
51. The composition of any one of the preceding embodiments, further comprising one or more pH-sensitive enteric polymers.
52. The composition of any one of the preceding embodiments, wherein each of the bacterial strains is present in an amount from 10⁵ to 10¹³ colony forming units (CFUs) per gram of the composition.
53. The composition of any one of the preceding embodiments, wherein each of the bacterial strains is present in an amount from 10⁵ to 10⁸ CFUs per gram of the composition.
54. A nutritional supplement comprising the composition of any one of the preceding embodiments, and a carrier.
55. The nutritional supplement of embodiment 54, wherein each of the bacterial strains is present in an amount from 10⁵ to 10¹³ colony forming units (CFUs) per gram of the nutritional supplement.
56. The nutritional supplement of any one of the preceding embodiments, wherein each of the bacterial strains is present in an amount from 10⁸ to 10¹⁰ CFUs per gram of the nutritional supplement.
57. The nutritional supplement of any one of the preceding embodiments, wherein the nutritional supplement has a water activity (a_{w}) of 0.8 or less.
58. The nutritional supplement of any one of the preceding embodiments, wherein at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the nutritional supplement are viable after storage for a period of time.
59. The nutritional supplement of any one of the preceding embodiments, wherein the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.
60. The nutritional supplement of any one of the preceding embodiments, wherein the storage is conducted at 2° C. to 8° C., 8° C. to 20° C., or 20° C. to 40° C. 61. The nutritional supplement of any one of the preceding embodiments, wherein the storage is conducted at a temperature of 25° C. or more.
62. The nutritional supplement of any one of the preceding embodiments, wherein the nutritional supplement has a water activity level (a_{w}) of 0.25 or more, 0.3 or more, 0.4 or more, 0.5 or more, 0.6 or more, or 0.7 or more during the storage.
63. The nutritional supplement of any one of the preceding embodiments, wherein the nutritional supplement is a canine nutritional supplement.
64. The nutritional supplement of any one of the preceding embodiments, wherein the nutritional supplement comprises a meat or animal-derived material.
65. The nutritional supplement of any one of the preceding embodiments, wherein the meat or animal-derived material is beef, pork, chicken, eggs, turkey, lamb, fish, blood marrow, and/or bone marrow.
66. The nutritional supplement of any one of the preceding embodiments, wherein the nutritional supplement comprises a grain.
67. The nutritional supplement of any one of the preceding embodiments, wherein the grain is wheat, corn, rice, oats, and/or barley.
68. The nutritional supplement of any one of the preceding embodiments, wherein the nutritional supplement comprises a fiber.
69. The nutritional supplement of any one of the preceding embodiments, wherein the fiber is sugar beet pulp, chicory pulp, chicory, coconut endosperm fiber, wheat fiber, fructooligosaccharides, galactooligosaccharides, and/or inulin.
70. The nutritional supplement of any one of the preceding embodiments, wherein the nutritional supplement comprises a gelatinized starch matrix.
71. The nutritional supplement of any one of the preceding embodiments, wherein the nutritional supplement reduces inflammation in a subject.
72. The nutritional supplement of any one of the preceding embodiments, wherein the nutritional supplement treats or prevents a symptom associated with inflammation in a subject.
73. The nutritional supplement of any one of the preceding embodiments, wherein the nutritional supplement treats or prevents diarrhea in a subject.
74. The nutritional supplement of any one of the preceding embodiments, wherein the nutritional supplement treats or prevents vomiting in a subject.
75. The nutritional supplement of any one of the preceding embodiments, wherein the nutritional supplement treats or prevents itching, paw licking, and/or head shaking in a subject.
76. A method for producing a nutritional supplement, the method comprising:
   (i) providing a carrier; and
   (ii) adding a composition comprising a purified bacterial mixture to the carrier to produce the nutritional supplement,
   wherein the purified bacterial mixture comprises:
      (a) a first bacterial strain comprising a 16S rDNA sequence having at least 97% sequence identity to the nucleic acid sequence of SEQ ID NO: 1;
      (b) a second bacterial strain comprising a 16S rDNA sequence having at least 97% sequence identity to the nucleic acid sequence of SEQ ID NO: 2; and
      (c) a third bacterial strain comprising a 16S rDNA sequence having at least 97% sequence identity to the nucleic acid sequence of SEQ ID NO: 3;
   wherein the first bacterial strain and third bacterial strain are different bacterial strains.
77. The method of embodiment 76, wherein the purified bacterial mixture consists of:
   (i) the first bacterial strain comprising a 16S rDNA sequence having at least 97% sequence identity to the nucleic acid sequence of SEQ ID NO: 1;
   (ii) the second bacterial strain comprising a 16S rDNA sequence having at least 97% sequence identity to the nucleic acid sequence of SEQ ID NO: 2; and
   (iii) the third bacterial strain comprising a 16S rDNA sequence having at least 97% sequence identity to the nucleic acid sequence of SEQ ID NO: 3.
78. The method of any one of the preceding embodiments, wherein
   (i) the first bacterial strain comprises a 16S rDNA sequence having the nucleic acid sequence of SEQ ID NO: 1;
   (ii) the second bacterial strain comprises a 16S rDNA sequence having the nucleic acid sequence of SEQ ID NO: 2; and
   (iii) the third bacterial strain comprises a 16S rDNA sequence having the nucleic acid sequence of SEQ ID NO: 3.
79. A method for producing a nutritional supplement, the method comprising:
   (i) providing a carrier; and
   (ii) adding a composition comprising a purified bacterial mixture to the carrier to produce the nutritional supplement,
   wherein the purified bacterial mixture comprises:
      (a) a first bacterial strain comprising a 16S rDNA sequence having at least 97% sequence identity to the nucleic acid sequence of SEQ ID NO: 4;
      (b) a second bacterial strain comprising a 16S rDNA sequence having at least 97% sequence identity to the nucleic acid sequence of SEQ ID NO: 5; and
      (c) a third bacterial strain comprising a 16S rDNA sequence having at least 97% sequence identity to the nucleic acid sequence of SEQ ID NO: 6;
   wherein the first bacterial strain and third bacterial strain are different bacterial strains.
80. The method of any one of the preceding embodiments, wherein the purified bacterial mixture consists of:
   (i) the first bacterial strain comprising a 16S rDNA sequence having at least 97% sequence identity to the nucleic acid sequence of SEQ ID NO: 4;
   (ii) the second bacterial strain comprising a 16S rDNA sequence having at least 97% sequence identity to the nucleic acid sequence of SEQ ID NO: 5; and
   (iii) the third bacterial strain comprising a 16S rDNA sequence having at least 97% sequence identity to the nucleic acid sequence of SEQ ID NO: 6.
81. The method of any one of the preceding embodiments, wherein
   (i) the first bacterial strain comprises a 16S rDNA sequence having the nucleic acid sequence of SEQ ID NO: 4;
   (ii) the second bacterial strain comprises a 16S rDNA sequence having the nucleic acid sequence of SEQ ID NO: 5; and
   (iii) the third bacterial strain comprises a 16S rDNA sequence having the nucleic acid sequence of SEQ ID NO: 6.
82. A method for producing a nutritional supplement, the method comprising:
   (i) providing a carrier; and
   (ii) adding a composition comprising a purified bacterial mixture to the carrier to produce the nutritional supplement,
   wherein the purified bacterial mixture comprises a first bacterial strain belonging to the species Bacillus subtilis, a second bacterial strain belonging to the species Bacillus velezensis, and a third bacterial strain belonging to the species Bacillus subtilis, wherein the first bacterial strain and the third bacterial strain are different bacterial strains.
83. The method of any one of the preceding embodiments, wherein the purified bacterial mixture consists of a first bacterial strain belong to the species Bacillus subtilis, a second bacterial strain belongs to the species Bacillus velezensis, and a third bacterial strain belong to the species Bacillus subtilis.
84. The method of any one of the preceding embodiments, wherein the purified bacterial mixture further comprises one or more additional bacterial strains.
85. The method of any one of the preceding embodiments, wherein one or more of the additional bacterial strains belong to the genus Bacillus.
86. The method of any one of the preceding embodiments, wherein one or more of the additional bacterial strains belong to the species Bacillus subtilis, Bacillus velezensis, Bacillus amyloliquefaciens, Bacillus lentus, Bacillus licheniformis, or Bacillus pumilus.
87. The method of any one of the preceding embodiments, wherein one or more of the additional bacterial strains comprise a 16S rDNA sequence with at least 97% sequence identity to the nucleic acid sequence of any one of SEQ ID NOs: 1-3.
88. The method of any one of the preceding embodiments, wherein one or more of the additional bacterial strains comprise a 16S rDNA sequence with at least 97% sequence identity to the nucleic acid sequence of any one of SEQ ID NOs; 4-6.
89. The method of any one of the preceding embodiments, wherein the composition produces one or more indole-containing molecules in the presence of L-tryptophan.
90. The method of any one of the preceding embodiments, wherein the composition produces at least 4 indole-containing molecules in the presence of L-tryptophan.
91. The method of any one of the preceding embodiments, wherein the indole-containing molecules comprise indoleacetic acid, indoleacrylic acid, indolepropionic acid, indolepyruvic acid, indoleacetamide, indole-3-acetic acid, indole-3-ethanol, indolealdehyde, indole, and/or tryptamine.
92. The method of any one of the preceding embodiments, wherein the composition produces indole-3-acetic acid in a concentration of 8.5 μM or more.
93. The method of any one of the preceding embodiments, wherein the composition produces indoleacetic acid in a concentration of 10 μM or more.
94. The method of any one of the preceding embodiments, wherein the composition produces indolepropionic acid in a concentration of 1.0 μM or more.
95. The method of any one of the preceding embodiments, wherein the composition produces indole-3-aldehyde in a concentration of 1.0 μM or more.
96. The method of any one of the preceding embodiments, wherein the composition produces L-tryptophan in the presence of protein.
97. The method of any one of the preceding embodiments, wherein the composition reduces inflammation in a subject.
98. The method of any one of the preceding embodiments, wherein the composition treats or prevents a symptom associated with inflammation in a subject.
99. The method of any one of the preceding embodiments, wherein the composition treats or prevents diarrhea in a subject.
100. The method of any one of the preceding embodiments, wherein the composition treats or prevents vomiting in a subject.
101. The method of any one of the preceding embodiments, wherein the composition treats or prevents itching, paw licking, and/or head shaking in a subject.
102. The method of any one of the preceding embodiments, wherein at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the composition are viable after storage for a period of time.
10³. The method of any one of the preceding embodiments, wherein the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.
104. The method of any one of the preceding embodiments, wherein the storage is conducted at 2° C. to 8° C., 8° C. to 20° C., or 20° C. to 40° C.
105. The method of any one of the preceding embodiments, wherein the storage is conducted at a temperature of 25° C. or more.
106. The method of any one of the preceding embodiments, wherein the composition has a water activity level (a_{w}) of 0.25 or more, 0.3 or more, 0.4 or more, 0.5 or more, 0.6 or more, or 0.7 or more during the storage.
107. The method of any one of the preceding embodiments, wherein the bacterial strains are lyophilized.
108. The method of any one of the preceding embodiments, wherein the bacterial strains are spray-dried.
109. The method of any one of the preceding embodiments, wherein one or more of the bacterial strains is in spore form.
110. The method of any one of the preceding embodiments, wherein each of the bacterial strains is in spore form.
111. The method of any one of the preceding embodiments, wherein one or more of the bacterial strains is in vegetative form.
112. The method of any one of the preceding embodiments, wherein each of the bacterial strains is in vegetative form.
113. The method of any one of the preceding embodiments, wherein the composition has a water activity level (a_{w}) of 0.8 or less.
114. The method of any one of the preceding embodiments, wherein the composition further comprises one or more pH-sensitive enteric polymers.
115. The method of any one of the preceding embodiments, wherein each of the bacterial strains is present in an amount from 10⁵ to 10¹³ colony forming units (CFUs) per gram of the composition.
116. The method of any one of the preceding embodiments, wherein each of the bacterial strains is present in an amount from 10⁵ to 10⁸ CFUs per gram of the nutritional supplement.
117. The method of any one of the preceding embodiments, wherein each of the bacterial strains is present in an amount from 10⁵ to 10¹³ colony forming units (CFUs) per gram of the nutritional supplement.
118. The method of any one of the preceding embodiments, wherein each of the bacterial strains is present in an amount from 10⁸ to 10¹⁰ CFUs per gram of the nutritional supplement.
119. The method of any one of the preceding embodiments, wherein the nutritional supplement has a water activity (a_{w}) of 0.8 or less.
120. The method of any one of the preceding embodiments, wherein at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the nutritional supplement are viable after storage for a period of time.
121. The method of any one of the preceding embodiments, wherein the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.
122. The method of any one of the preceding embodiments, wherein the storage is conducted at 2° C. to 8° C., 8° C. to 20° C., or 20° C. to 40° C. 123. The method of any one of the preceding embodiments, wherein the storage is conducted at a temperature of 25° C. or more.
124. The method of any one of the preceding embodiments, wherein the nutritional supplement has a water activity level (a_{w}) of 0.25 or more, 0.3 or more, 0.4 or more, 0.5 or more, 0.6 or more, or 0.7 or more during the storage.
125. The method of any one of the preceding embodiments, wherein the nutritional supplement is a canine nutritional supplement.
126. The method of any one of the preceding embodiments, wherein the nutritional supplement comprises a meat or animal-derived material.
127. The method of any one of the preceding embodiments, wherein the meat or animal-derived material is beef, pork, chicken, eggs, turkey, lamb, fish, blood marrow, and/or bone marrow.
128. The method of any one of the preceding embodiments, wherein the nutritional supplement comprises a grain.
129. The method of any one of the preceding embodiments, wherein the grain is wheat, corn, rice, oats, and/or barley.
130. The method of any one of the preceding embodiments, wherein the nutritional supplement comprises a fiber.
132. The method of any one of the preceding embodiments, wherein the fiber is sugar beet pulp, chicory pulp, chicory, coconut endosperm fiber, wheat fiber, fructooligosaccharides, galactooligosaccharides, and/or inulin.
133. The method of any one of the preceding embodiments, wherein the nutritional supplement comprises a gelatinized starch matrix.
134. The method of any one of the preceding embodiments, wherein the nutritional supplement comprises a humectant.
135. The method of any one of the preceding embodiments, wherein the nutritional supplement reduces inflammation in a subject.
136. The method of any one of the preceding embodiments, wherein the nutritional supplement treats or prevents a symptom associated with inflammation in a subject.
137. The method of any one of the preceding embodiments, wherein the nutritional supplement treats or prevents diarrhea in a subject.
138. The method of any one of the preceding embodiments, wherein the nutritional supplement treats or prevents vomiting in a subject.
139. The method of any one of the preceding embodiments, wherein the nutritional supplement treats or prevents itching, paw licking, and/or head shaking in a subject.
140. The method of any one of the preceding embodiments, wherein the nutritional supplement treats or prevents one or more symptoms associated with allergy in a subject.
141. A nutritional supplement produced by the method of any one of the preceding embodiments.
142. A food product comprising the composition of any one of the preceding embodiments and a nutrient.
143. The food product of embodiment 142, wherein each of the bacterial strains is present in an amount from 10⁵ to 10¹³ colony forming units (CFUs) per gram of the food product.
144. The food product of any one of the preceding embodiments, wherein each of the bacterial strains is present in an amount from 10⁸ to 10¹⁰ CFUs per gram of the food product.
145. The food product of any one of the preceding embodiments, wherein the food product has a water activity (a_{w}) of 0.8 or less.
146. The food product of any one of the preceding embodiments, wherein at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the food product are viable after storage for a period of time.
147. The food product of any one of the preceding embodiments, wherein the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.
148. The food product of any one of the preceding embodiments, wherein the storage is conducted at 2° C. to 8° C., 8° C. to 20° C., or 20° C. to 40° C.
149. The food product of any one of the preceding embodiments, wherein the storage is conducted at a temperature of 25° C. or more.
150. The food product of any one of the preceding embodiments, wherein the food product has a water activity level (a_{w}) of 0.25 or more, 0.3 or more, 0.4 or more, 0.5 or more, 0.6 or more, or 0.7 or more during the storage.
151. The food product of any one of the preceding embodiments, wherein the food product is a canine food product.
152. The food product of any one of the preceding embodiments, wherein the food product comprises a meat or animal-derived material.
153. The food product of any one of the preceding embodiments, wherein the meat or animal-derived material is beef, chicken, eggs, turkey, lamb, fish, blood marrow, and/or bone marrow.
154. The food product of any one of the preceding embodiments, wherein the food product comprises a grain.
155. The food product of any one of the preceding embodiments, wherein the grain is wheat, corn, rice, oats, and/or barley.
156. The food product of any one of the preceding embodiments, wherein the food product comprises a fiber.
157. The food product of any one of the preceding embodiments, wherein the fiber is sugar beet pulp, chicory pulp, chicory, coconut endosperm fiber, wheat fiber, fructooligosaccharides, galactooligosaccharides, and/or inulin.
158. The food product of any one of the preceding embodiments, wherein the food product comprises a gelatinized starch matrix.
159. The food product of any one of the preceding embodiments, wherein the food product comprises a humectant.
160. The food product of any one of the preceding embodiments, wherein the food product reduces inflammation in a subject.
161. The food product of any one of the preceding embodiments, wherein the food product treats or prevents a symptom associated with inflammation in a subject.
162. The food product of any one of the preceding embodiments, wherein the food product treats or prevents diarrhea in a subject.
163. The food product of any one of the preceding embodiments, wherein the food product treats or prevents vomiting in a subject.
164. The food product of any one of the preceding embodiments, wherein the food product treats or prevents itching, paw licking, and/or head shaking in a subject.
165. A method for producing a food product, the method comprising:
   (i) providing a carrier; and
   (ii) adding a composition comprising a purified bacterial mixture to the nutrient to produce the food product,
   wherein the purified bacterial mixture comprises:
      (a) a first bacterial strain comprising a 16S rDNA sequence having at least 97% sequence identity to the nucleic acid sequence of SEQ ID NO: 1;
      (b) a second bacterial strain comprising a 16S rDNA sequence having at least 97% sequence identity to the nucleic acid sequence of SEQ ID NO: 2; and
      (c) a third bacterial strain comprising a 16S rDNA sequence having at least 97% sequence identity to the nucleic acid sequence of SEQ ID NO: 3;
   wherein the first bacterial strain and third bacterial strain are different bacterial strains.
166. The method of embodiment 165, wherein the purified bacterial mixture consists of:
   (i) the first bacterial strain comprising a 16S rDNA sequence having at least 97% sequence identity to the nucleic acid sequence of SEQ ID NO: 1;
   (ii) the second bacterial strain comprising a 16S rDNA sequence having at least 97% sequence identity to the nucleic acid sequence of SEQ ID NO: 2; and
   (iii) the third bacterial strain comprising a 16S rDNA sequence having at least 97% sequence identity to the nucleic acid sequence of SEQ ID NO: 3.
167. The method of any one of the preceding embodiments, wherein
   (i) the first bacterial strain comprises a 16S rDNA sequence having the nucleic acid sequence of SEQ ID NO: 1;
   (ii) the second bacterial strain comprises a 16S rDNA sequence having the nucleic acid sequence of SEQ ID NO: 2; and
   (iii) the third bacterial strain comprises a 16S rDNA sequence having the nucleic acid sequence of SEQ ID NO: 3.
168. A method for producing a food product, the method comprising:
   (i) providing a carrier; and
   (ii) adding a composition comprising a purified bacterial mixture to the carrier to produce the food product,
   wherein the purified bacterial mixture comprises:
      (a) a first bacterial strain comprising a 16S rDNA sequence having at least 97% sequence identity to the nucleic acid sequence of SEQ ID NO: 4;
      (b) a second bacterial strain comprising a 16S rDNA sequence having at least 97% sequence identity to the nucleic acid sequence of SEQ ID NO: 5; and
      (c) a third bacterial strain comprising a 16S rDNA sequence having at least 97% sequence identity to the nucleic acid sequence of SEQ ID NO: 6;
   wherein the first bacterial strain and third bacterial strain are different bacterial strains.
169. The method of embodiment 168, wherein the purified bacterial mixture consists of:
   (i) the first bacterial strain comprising a 16S rDNA sequence having at least 97% sequence identity to the nucleic acid sequence of SEQ ID NO: 4;
   (ii) the second bacterial strain comprising a 16S rDNA sequence having at least 97% sequence identity to the nucleic acid sequence of SEQ ID NO: 5; and
   (iii) the third bacterial strain comprising a 16S rDNA sequence having at least 97% sequence identity to the nucleic acid sequence of SEQ ID NO: 6.
170. The method of any one of the preceding embodiments, wherein
   (i) the first bacterial strain comprises a 16S rDNA sequence having the nucleic acid sequence of SEQ ID NO: 4;
   (ii) the second bacterial strain comprises a 16S rDNA sequence having the nucleic acid sequence of SEQ ID NO: 5; and
   (iii) the third bacterial strain comprises a 16S rDNA sequence having the nucleic acid sequence of SEQ ID NO: 6.
171. A method for producing a food product, the method comprising:
   (i) providing a nutrient; and
   (ii) adding a composition comprising a purified bacterial mixture to the nutrient to produce the food product,
   wherein the purified bacterial mixture comprises a first bacterial strain belonging to the species Bacillus subtilis, a second bacterial strain belonging to the species Bacillus velezensis, and a third bacterial strain belonging to the species Bacillus subtilis, wherein the first bacterial strain and the third bacterial strain are different bacterial strains.
172. The method of any one of the preceding embodiments, wherein the purified bacterial mixture consists of a first bacterial strain belong to the species Bacillus subtilis, a second bacterial strain belongs to the species Bacillus velezensis, and a third bacterial strain belong to the species Bacillus subtilis.
173. The method of any one of the preceding embodiments, wherein the purified bacterial mixture further comprises one or more additional bacterial strains.
174. The method of any one of the preceding embodiments, wherein one or more of the additional bacterial strains belong to the genus Bacillus.
175. The method of any one of the preceding embodiments, wherein one or more of the additional bacterial strains belong to the species Bacillus subtilis, Bacillus velezensis, Bacillus amyloliquefaciens, Bacillus lentus, Bacillus licheniformis, or Bacillus pumilus.
176. The method of any one of the preceding embodiments, wherein one or more of the additional bacterial strains comprise a 16S rDNA sequence with at least 97% sequence identity to the nucleic acid sequence of any one of SEQ ID NOs: 1-3.
177 The method of any one of the preceding embodiments, wherein one or more of the additional bacterial strains comprise a 16S rDNA sequence with at least 97% sequence identity to the nucleic acid sequence of any one of SEQ ID NOs; 4-6.
178. The method of any one of the preceding embodiments, wherein the composition produces one or more indole-containing molecules in the presence of L-tryptophan.
179. The method of any one of the preceding embodiments, wherein the composition produces at least 4 indole-containing molecules in the presence of L-tryptophan.
180. The method of any one of the preceding embodiments, wherein the indole-containing molecules comprise indoleacetic acid, indoleacrylic acid, indolepropionic acid, indolepyruvic acid, indoleacetamide, indole-3-acetic acid, indolealdehyde, indole, and/or tryptamine.
181. The method of any one of the preceding embodiments, wherein the composition produces indole-3-acetic acid in a concentration of 8.5 UM or more.
182. The method of any one of the preceding embodiments, wherein the composition produces indoleacetic acid in a concentration of 10 μM or more.
183. The method of any one of the preceding embodiments, wherein the composition produces indolepropionic acid in a concentration of 1.0 UM or more.
184. The method of any one of the preceding embodiments, wherein the composition produces indole-3-aldehyde in a concentration of 1.0 μM or more.
185. The method of any one of the preceding embodiments, wherein the composition produces L-tryptophan in the presence of protein.
186. The method of any one of the preceding embodiments, wherein the composition reduces inflammation in a subject.
187. The method of any one of the preceding embodiments, wherein the composition treats or prevents a symptom associated with inflammation in a subject.
188. The method of any one of the preceding embodiments, wherein the composition treats or prevents diarrhea in a subject.
189. The method of any one of the preceding embodiments, wherein the composition treats or prevents vomiting in a subject.
190. The method of any one of the preceding embodiments, wherein the composition treats or prevents itching, paw licking, and/or head shaking in a subject.
191. The method of any one of the preceding embodiments, wherein at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the composition are viable after storage for a period of time.
192. The method of any one of the preceding embodiments, wherein the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.
193. The method of any one of the preceding embodiments, wherein the storage is conducted at 2° C. to 8° C., 8° C. to 20° C., or 20° C. to 40° C.
194. The method of any one of the preceding embodiments, wherein the storage is conducted at a temperature of 25° C. or more.
195. The method of any one of the preceding embodiments, wherein the composition has a water activity level (a_{w}) of 0.25 or more, 0.3 or more, 0.4 or more, 0.5 or more, 0.6 or more, or 0.7 or more during the storage.
196. The method of any one of the preceding embodiments, wherein the bacterial strains are lyophilized.
197. The method of any one of the preceding embodiments, wherein the bacterial strains are spray-dried.
198. The method of any one of the preceding embodiments, wherein one or more of the bacterial strains is in spore form.
199. The method of any one of the preceding embodiments, wherein each of the bacterial strains is in spore form.
200. The method of any one of the preceding embodiments, wherein one or more of the bacterial strains is in vegetative form.
201. The method of any one of the preceding embodiments, wherein each of the bacterial strains is in vegetative form.
202. The method of any one of the preceding embodiments, wherein the food product has a water activity level (a_{w}) of 0.8 or less.
203. The method of any one of the preceding embodiments, wherein the food product further comprises one or more pH-sensitive enteric polymers.
204. The method of any one of the preceding embodiments, wherein each of the bacterial strains is present in an amount from 10⁵ to 10¹³ colony forming units (CFUs) per gram of the food product.
205. The method of any one of the preceding embodiments, wherein each of the bacterial strains is present in an amount from 10⁵ to 10⁸ CFUs per gram of the food product.
206. The method of any one of the preceding embodiments, wherein each of the bacterial strains is present in an amount from 10⁵ to 10¹³ colony forming units (CFUs) per gram of the food product.
207. The method of any one of the preceding embodiments, wherein each of the bacterial strains is present in an amount from 10⁸ to 10¹⁰ CFUs per gram of the food product.
208. The method of any one of the preceding embodiments, wherein the food product has a water activity (a_{w}) of 0.8 or less.
209. The method of any one of the preceding embodiments, wherein at least 10%, at least 20%, at least 30%, at least 40%, at least 50%, at least 60%, at least 70%, at least 75%, at least 80%, at least 85%, at least 95%, at least 97%, at least 98%, at least 99%, or at least 100% of colony forming units in the food product are viable after storage for a period of time.
210. The method of any one of the preceding embodiments, wherein the period of time is at least 1 week, at least 2 weeks, at least 3 weeks, at least 4 weeks, at least 6 weeks, at least 8 weeks, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.
211. The method of any one of the preceding embodiments, wherein the storage is conducted at 2° C. to 8° C., 8° C. to 20° C., or 20° C. to 40° C. 212. The method of any one of any one of the preceding embodiments, wherein the storage is conducted at a temperature of 25° C. or more.
213. The method of any one of the preceding embodiments, wherein the food product has a water activity level (a_{w}) of 0.25 or more, 0.3 or more, 0.4 or more, 0.5 or more, 0.6 or more, or 0.7 or more during the storage.
214. The method of any one of the preceding embodiments, wherein the food product is a canine food product.
215. The method of any one of the preceding embodiments, wherein the food product comprises a meat or animal-derived material.
216. The method of any one of the preceding embodiments, wherein the meat or animal-derived material is beef, chicken, eggs, turkey, lamb, fish, blood marrow, and/or bone marrow.
217. The method of any one of the preceding embodiments, wherein the food product comprises a grain.
218. The method of any one of the preceding embodiments, wherein the grain is wheat, corn, rice, oats, and/or barley.
219. The method of any one of the preceding embodiments, wherein the food product comprises a fiber.
220. The method of any one of the preceding embodiments, wherein the fiber is sugar beet pulp, chicory pulp, chicory, coconut endosperm fiber, wheat fiber, fructooligosaccharides, galactooligosaccharides, and/or inulin.
221. The method of any one of the preceding embodiments, wherein the food product comprises a gelatinized starch matrix.
222. The method of any one of the preceding embodiments, wherein the food product reduces inflammation in a subject.
223. The method of any one of the preceding embodiments, wherein the food product treats or prevents a symptom associated with inflammation in a subject.
224. The method of any one of the preceding embodiments, wherein the food product treats or prevents diarrhea in a subject.
225. The method of any one of the preceding embodiments, wherein the food product treats or prevents vomiting in a subject.
226. The method of any one of the preceding embodiments, wherein the food product treats or prevents itching, paw licking, and/or head shaking in a subject.
227. The method of any one of the preceding embodiments, wherein the food product treats or prevents one or more symptoms associated with allergy in a subject.
228. A food product produced by the method of any one of the preceding embodiments.
229. A method comprising administering the composition, nutritional supplement, food product of any one of the preceding embodiments, to a subject in need thereof.
230. The method of embodiment 229, wherein the subject is a carnivore.
231. The method of any one of the preceding embodiments, wherein the subject is a mammal.
232. The method of any one of the preceding embodiments, wherein the subject is a domesticated animal.
233. The method of any one of the preceding embodiments, wherein the subject is a rodent.
234. The method of any one of the preceding embodiments, wherein the subject is a mouse, rat, guinea pig, chinchilla, or hamster.
235. The method of any one of the preceding embodiments, wherein the subject is a dog, cat, rabbit, guinea pig, hamster, or ferret.
236. The method of any one of the preceding embodiments, wherein the subject is a dog.
237. The method of any one of the preceding embodiments, wherein the subject is a bovine, swine, llama, alpaca, sheep, or goat.
238. The method of any one of the preceding embodiments, wherein the subject is a bird.
239. The method of any one of the preceding embodiments, wherein the subject has inflammation, diarrhea, vomiting, itching, paw licking, and/or head shaking.
240. The method of any one of the preceding embodiments, wherein the subject has one or more symptoms associated with allergy.

### EXAMPLES

### Example 1: Identification of Bacterial Strains Catabolizing L-Tryptophan to Produce Indole-Containing Metabolites

To identify bacterial strains capable of producing indole-containing molecules of interest, a high-throughput quantitative screen of isolated bacterial strains was conducted to evaluate production of known indole-containing molecules derived from free L-tryptophan (FIG. 1A).

Using high-throughput liquid handlers, strains were grown in oxygen-limited conditions, and strain growth was measured by optical density (OD600) spectrophotometry to evaluate the ability of strains to propagate in these conditions (FIG. 1B). Next, subsequent 5-day fermentations of bacterial strains that showed high growth potential were carried out in a conventional rich media under microaerobic environment at 38° C. Resulting fermentation broths were subjected to liquid chromatography-high-resolution mass spectrometry to measure the abundance of indole-containing AhR agonists (FIG. 1C).

In brief, the measurements of these molecules were carried out by performing 1:1 organic solvent extractions of fermentation broth acidified using 1% formic acid, and injecting these extracts onto a reversed-phase chromatography set before a high-resolution quadrupole time-of-flight mass spectrometer (QTOF). Isotopically labeled internal standards were used to correct for sample preparation and data acquisition abnormalities. Standard curves were used to calculate concentrations with parent ion quantifiers and at least 2 qualifying MS² fragments to confirm structural identity of the molecular features. Quantification of metabolite production in the initial hits were validated by repeating high-throughput fermentations and AhR agonist measurements.

Bacterial strains that produced high levels of AhR agonists were evaluated to determine their capacity to form endospores. A combination of three spore-forming strains (Bacillus subtilis having a 16S rDNA sequence of SEQ ID NO: 1, Bacillus velezensis having a 16S rDNA sequence of SEQ ID NO: 2, and Bacillus subtilis having a 16S rDNA sequence of SEQ ID NO: 3), was used in larger scale fermentation (50-100 mL) under the same conditions as described above, to measure concentrations of AhR agonists produced by this community (FIG. 1D). The concentrations of indole-containing molecules were also measured following fermentation by individual strains. Strains 1 produced substantial concentrations of multiple different molecules, including ILA, IAId, IAA, and IPA, whereas Strain 2 produced a large concentration of ILA and IAA, and Strain 3 produced ILA and IAA (FIGS. 1E, 1G, and 1H). Strains were also fermented in media containing complex protein, to determine their ability to catabolize protein and produce free L-tryptophan for metabolism into indole-containing molecules. Strain #3 was shown to produce substantial amounts of L-tryptophan, allowing abundant substrate for production of indole-containing molecules by itself and other bacteria of a mixture (FIG. 1F, arrow).

Next, strains were fermented in cross-feeding containers, where two distinct bacterial populations were incubated in liquid medium on different sides of a semi-permeable membrane. Fermentation in this manner allowed exchange of small molecules by diffusion, but prevented contact between bacterial populations. Indoleacetic acid was produced when both populations belonged to Strain 1, or when both populations belonged to Strain 2. However, a markedly higher concentration of indoleacetic acid was observed when one population belonged to Strain 1 and the other belonged to Strain 2 (FIG. 2). These results indicate synergy in indole-containing molecule production between bacterial strains.

### Example 2: Stability of Bacterial Strains

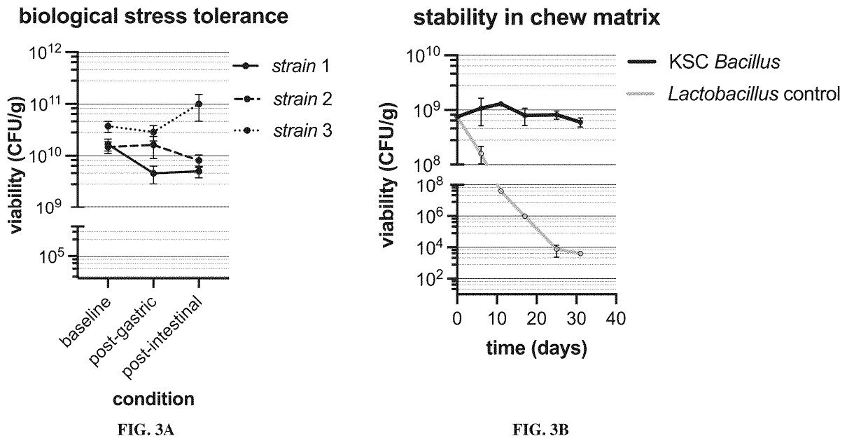

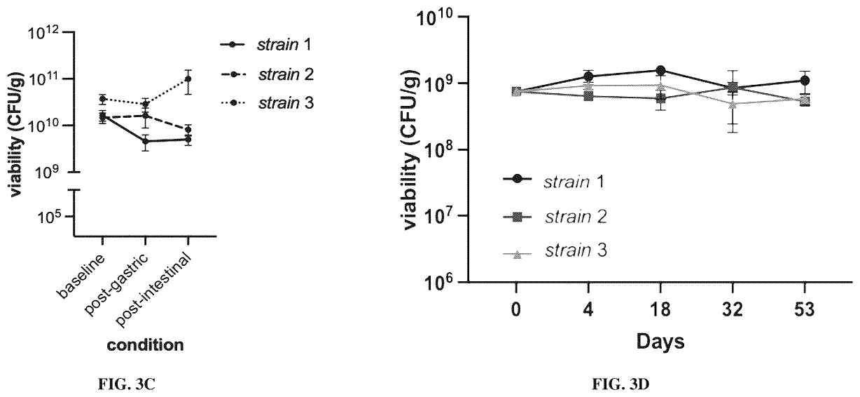

Bacteria were evaluated for viability in conditions similar to the gastrointestinal tract. Each strain was subjected to an environment mimicking gastric stress (pH 2, with high concentrations of endopeptidases), or intestinal stress (neutral pH, with endopeptidases and 18 g/L bile salts). Each of Strains 1-3 survived in these simulated gastric or intestinal environments, with reductions in viability being less than <0.5 log 10 (FIGS. 3A and 3C).

Stability of Bacillus Strains 1-3 was measured in a pet chew matrix with water activity (a_{w}) level of 0.4, which is typically growth-prohibitive for other bacteria such as Lactobacillus. While Lactobacillus viability declined over 4 weeks of storage, Bacillus Strains 1-3 remained viable for 4 weeks, with loss of viability being less than <0.5 log 10 (FIGS. 3B and 3D). These results indicate that Bacillus Strains 1-3 are stable in compositions having higher water content required to avoid undesired dry or brittle textures, as well as in environments resembling the gastrointestinal tract, making them suitable for delivery to, and production of indole-containing molecules in, the intestine.

### Example 3: Production of Indole-Containing Compounds by Bacterial Strains

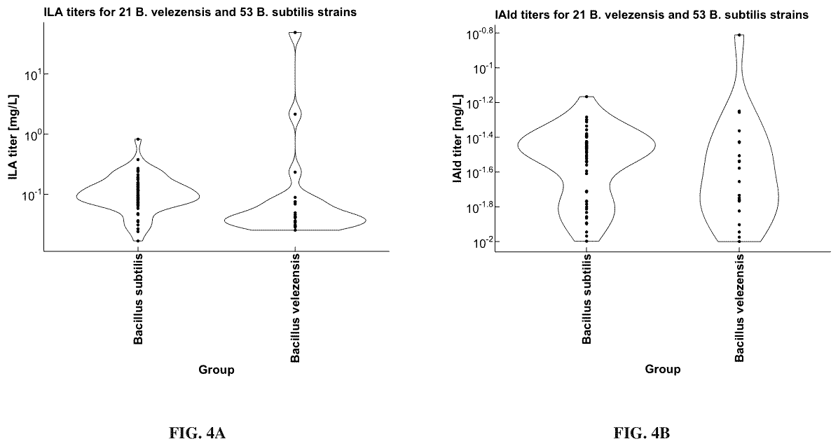

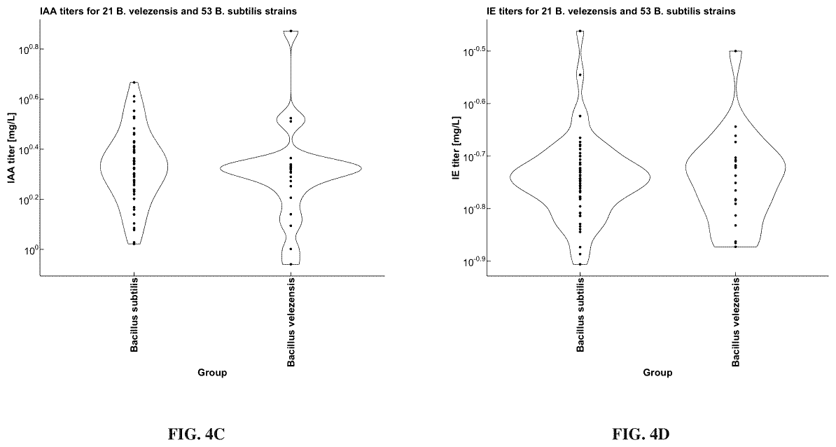

21 strains of Bacillus velezensis and 53 strains of Bacillus subtilis were evaluated individually for production of indole-containing compounds, including indole-3-lactic acid (ILA), indole-3-aldehyde (IAld), indole-3-acetic acid (IAA), and tryptophol (IE). The results of these experiments are shown in FIGS. 4A-4D. Both species produced substantial titers of ILA, IAld, IAA, and IE.

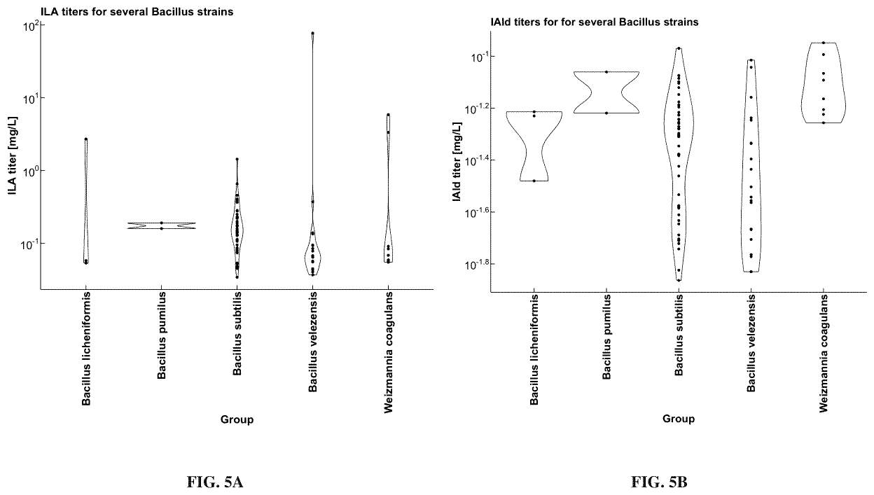

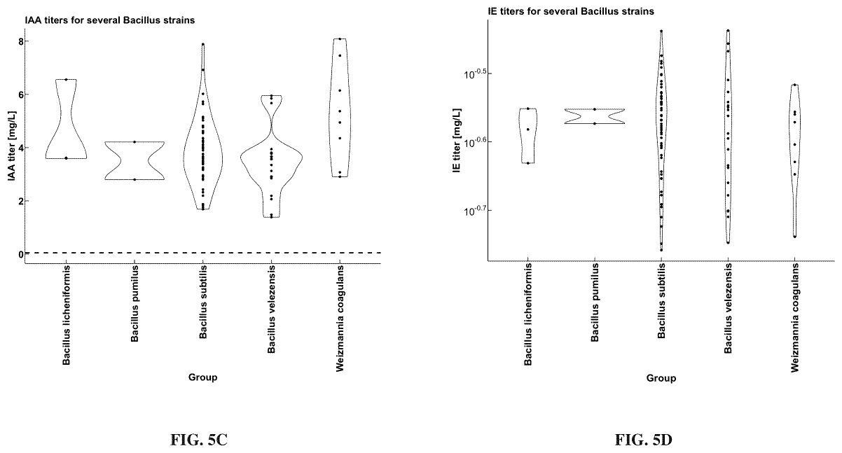

Production of ILA, IAld, IAA, and IE by other bacterial species, including Bacillus licheniformis, Bacillus pumilus, and Weizmannia coagulans, was also evaluated. The results of these experiments are shown in FIGS. 5A-5D. Each species produced ILA, IAld, IAA, and IE, indicating that Bacillus licheniformis, Bacillus pumilus, and Weizmannia coagulans are also suitable for production of indole-containing compounds.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A nutritional supplement comprising a bacterial mixture and a carrier, the bacterial mixture comprising:
(i) a first bacterial strain comprising a 16S rDNA sequence with at least 97% sequence identity to the nucleotide sequence of SEQ ID NO: 1;
(ii) a second bacterial strain comprising a 16S rDNA sequence with at least 97% sequence identity to the nucleotide sequence of SEQ ID NO: 2; and
(iii) a third bacterial strain comprising a 16S rDNA sequence with at least 97% sequence identity to the nucleotide sequence of SEQ ID NO: 3;
wherein the first and third bacterial strains are different bacterial strains; and
wherein the carrier comprises a gelatinized starch matrix, and the nutritional supplement has a water activity (a_{w}) of 0.25 or more.

2. The nutritional supplement of claim 1, wherein the water activity (a_{w}) is between 0.4 to 0.7.

3. The nutritional supplement of claim 1, wherein the carrier comprises maltodextrin.

4. The nutritional supplement of claim 1, wherein the carrier comprises a starch and/or a fiber, optionally a soluble fiber or an insoluble fiber.

5. The nutritional supplement of claim 4, wherein the fiber is selected from the group consisting of sugar beet pulp, chicory pulp, chicory, coconut endosperm fiber, wheat fiber, fructooligosaccharides, galactooligosaccharides, and inulin.

6. The nutritional supplement of claim 1, wherein the nutritional supplement is in the form of a chew.

7. The nutritional supplement of claim 1, wherein the bacterial strains are lyophilized or spray-dried.

8. The nutritional supplement of claim 1, wherein the bacterial strains are in spore form.

9. The nutritional supplement of claim 1, wherein the bacterial mixture consists of:
(i) the first bacterial strain comprising a 16S rDNA sequence with at least 97% sequence identity to the nucleotide sequence of SEQ ID NO: 1;
(ii) the second bacterial strain comprising a 16S rDNA sequence with at least 97% sequence identity to the nucleotide sequence of SEQ ID NO: 2; and
(iii) the third bacterial strain comprising a 16S rDNA sequence with at least 97% sequence identity to the nucleotide sequence of SEQ ID NO: 3.

10. The nutritional supplement of claim 1, wherein the bacterial mixture comprises:
(i) the first bacterial strain comprising a 16S rDNA sequence with at least 99% sequence identity to the nucleotide sequence of SEQ ID NO: 1;
(ii) the second bacterial strain comprising a 16S rDNA sequence with at least 99% sequence identity to the nucleotide sequence of SEQ ID NO: 2; and
(iii) the third bacterial strain comprising a 16S rDNA sequence with at least 99% sequence identity to the nucleotide sequence of SEQ ID NO: 3.

11. The nutritional supplement of claim 1, wherein the nutritional supplement comprises 1×10⁷ to 1×10⁹ colony forming units (CFUs) of each of the bacterial strains per gram of the nutritional supplement.

12. The nutritional supplement of claim 1, wherein the nutritional supplement comprises 1×10⁸ to 1×10¹⁰ total colony forming units (CFUs) of the bacterial strains per gram of the nutritional supplement.

13. The nutritional supplement of claim 1, wherein at least 50% of colony forming units of the bacterial strains are viable after storage for 4 weeks.

14. The nutritional supplement of claim 13, wherein the storage is conducted at a temperature of at least 25° C.

15. The nutritional supplement of claim 1, wherein the nutritional supplement produces indole-3-lactic acid (ILA), indole-3-aldehyde (IAld), and indole-3-acetic acid (IAA) in a subject.

16. The nutritional supplement of claim 15, wherein the ILA, IAld, and IAA are produced in an intestine of the subject.

17. The nutritional supplement of claim 1, wherein the nutritional supplement treats or prevents itching, paw licking, head shaking, and/or allergy in a subject.

18. The nutritional supplement of claim 15, wherein the subject is a dog.

19. The nutritional supplement of claim 1, wherein the nutritional supplement comprises a grain selected from the group consisting of wheat, corn, rice, oats, and barley.

20. The nutritional supplement of claim 1, wherein the nutritional supplement comprises a meat, optionally an animal-derived material selected from the group consisting of beef, chicken, pork, eggs, turkey, lamb, fish, blood marrow, and bone marrow.


SEQUENCE LISTING (6 sequences)

SEQ ID NO: 1 - DNA, 1525 bp, Bacillus subtilis
  source 1..1525: mol_type = genomic DNA
aggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 2 - DNA, 1351 bp, unidentified
  source 1..1351: mol_type = genomic DNA; note = Bacillus velezensis
agacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 3 - DNA, 1517 bp, Bacillus subtilis
  source 1..1517: mol_type = genomic DNA
cgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 4 - DNA, 278 bp, Bacillus subtilis
  source 1..278: mol_type = genomic DNA
gtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattaga

SEQ ID NO: 5 - DNA, 278 bp, unidentified; identical to SEQ ID NO: 4
  source 1..278: mol_type = genomic DNA; note = Bacillus velezensis

SEQ ID NO: 6 - DNA, 278 bp, Bacillus subtilis; identical to SEQ ID NO: 4
  source 1..278: mol_type = genomic DNA